(19) United States
(12) Patent Application Publication
(10) Pub. No.: US 2025/0000875 A1
(43) Pub. Date: Jan. 2, 2025

(54) ALLOPREGNANOLONE ANALOGUES FOR HIV VIREMIA AND NEUROTOXICITY PROTECTION

(71) Applicant: University of Mississippi, University, MS (US)

(72) Inventors: Hoang Le, Oxford, MS (US); Jason Paris, Little Rock, AR (US); Fakhri Mahdi, Oxford, MS (US); Md Imdadul H. Khan, Oxford, MS (US)

(21) Appl. No.: 18/684,743

(22) PCT Filed: Aug. 19, 2022
(86) PCT No.: PCT/US2022/075220

§ 371 Date: Feb. 19, 2024

Related U.S. Application Data
(60) Provisional application No. 63/235,435, filed on Aug. 20, 2021.

(51) Int. Cl. A61K 31/573; A61P 31/18; C07J 7/00; C07J 41/00

(52) U.S. Cl. CPC A61K 31/573; A61P 31/18; C07J 7/009; C07J 41/0005

(57) ABSTRACT

In one aspect. the disclosure relates to pregnane neurosteroid analogues of 5α-pregnan-3α-ol-20-one (3α,5α-THP). Also disclosed herein are methods of making the same, pharmaceutical compositions comprising the same, and methods of treating HIV and/or neuroHIV using the same. The pharmaceutical compositions can alleviate at least one symptom associated with HIV and/or neuroHIV and can be used alone or in combination with other HIV therapies including combination antiretroviral therapy (cART).

### CROSS REFERENCE TO RELATED APPLICATIONS

This application claims the benefit of U.S. Provisional Application No. 63/235,435 filed on Aug. 20, 2021, which is incorporated herein by reference in its entirety.

### BACKGROUND

About 1.2 million people in the United States are currently living with human immunodeficiency virus (HIV). Among patients infected with HIV-1, combination antiretroviral therapy (cART) has greatly reduced acquired immunodeficiency syndrome (AIDS) risk. However, infected individuals contend with pervasive HIV-associated neurocognitive and neuropsychiatric disorders, including deficits in learning and memory, behavioral inhibition, and affective well-being (hereafter, collectively referred to as “neuroHIV”). cART cannot eradicate neuroHIV, given that it poorly accumulates in the central nervous system (CNS) and does not target latent CNS viral reservoirs (microglia/macrophages and astrocytes). As such, ˜50% of HIV+ patients suffer from neuro-cognitive and-psychiatric disorders.

Estimates vary, but a considerable proportion (˜25%) of aviremic HIV-1 patients demonstrate dysfunction of the hypothalamic-pituitary-adrenal (HPA) stress axis (FIG. 1). In the pre-cART era, HPA dysfunction was largely “primary” (i.e. direct degeneration of the adrenals in >50% of patients). In the post-cART era, HPA dysfunction is largely “secondary” (i.e. mediated at the level of the CNS), likely due to cART's inability to control CNS viremia. HIV-mediated secondary HPA dysfunction is characterized by elevated glucocorticoid levels (e.g. cortisol) at baseline and (seemingly paradoxical) adrenal insufficiency in response to a stressor. This may be due to an HIV-mediated glucocorticoid insensitivity. The HPA axis plays a crucial role in an organism's ability to overcome physiological, psychological, and even immune stressors. HPA factors exert broad, pleiotropic effects to modulate cytokine profiles, central excitation, the peripheral stress response, etc. Elevated basal glucocorticoids, or adrenal insufficiency, exacerbate anxiety, depression, neurotoxicity, and reduce cognitive performance, symptoms of neuroHIV.

The mechanisms of neuroHIV are thought to involve neurotoxic viral proteins. The best characterized of these are the trans-activator of transcription (Tat) and glycoprotein 120 (gp120). Tat is a multifunctional, viral regulatory protein that drives HIV-1 transcription. It is present in post-mortem brain tissues and its expression persists in the CSF of aviremic HIV-1 patients despite CART. Tat promotes neuronal damage and excitotoxicity by direct or indirect activation of cation channels and proinflammatory cytokine release, and can disrupt ion homeostasis through activation of NMDA receptors and calcium channels, where increased Ca²⁺ can result in translocation of pro-apoptotic BAX and cytochrome c to cause neuronal damage. Tat further dysregulates the hypothalamic-pituitary-adrenal (HPA) stress axis to promote cognitive and affective dysfunction. These effects occur alone or in concert with gp120, the HIV-1 coat protein. Shed gp120 activates chemokine receptors, cation channels, and promotes neuroinflammation. Both proteins disrupt mitochondrial function, creating bioenergetic crisis. Using animal models, it has been found that Tat and/or gp120 to recapitulate a neuroHIV-like phenotype, increasing anxiety- and depression-like behavior, behavioral disinhibition, and cognitive deficits. Recent findings reveal Tat to produce glucocorticoid insensitivity in cultured splenocytes (FIG. 3). Moreover, transgenic Tat expression in mice recapitulates the clinical HPA axis phenotype, with elevated hypothalamic corticotropin releasing hormone (CRH) (FIG. 4) and circulating glucocorticoids (FIG. 5) at baseline, but adrenal insufficiency in response to glucocorticoid receptor blockade (FIG. 5). Some researchers have found gp120 sufficient to activate the HPA axis in mice. These data implicate Tat and gp120 in the pathogenesis of HPA axis dysfunction and related CNS complications.

A pregnane neurosteroid (allopregnanolone, also known as 5α-pregnan-3α-ol-20-one, alloP, or 3α,5α-THP), exerts anti-HIV-1 protein effects. 3α,5α-THP has been FDA-approved for other indications (i.e. brexanolone, Zulresso™), but suffers from rapid tissue re-distribution and a short plasma half-life. Accordingly, 3α,5α-THP is not likely to be druggable outside of a hospital where it can be administered intravenously. Furthermore, 3α,5α-THP is expensive and not efficacious in clinical use due to the short half-life, among other factors. Given that steroids are small, blood-brain barrier (BBB)-penetrant molecules with neuroprotective and anti-HIV-1 efficacy, however, 3α,5α-THP may be useful as a scaffold in the development of adjuncts to cART

The incidence of neuroHIV has not changed since the pre-cART era. cART cannot ameliorate the CNS complications of HIV-1, including HPA dysregulation and related cognitive/psychiatric effects. Adjunctive therapeutics that target CNS pathology are needed to finally reduce the incidence of neuroHIV. Ideally, these therapeutics would have broad applicability for a range of neurodegenerative diseases. These needs and other needs are satisfied by the present disclosure.

### SUMMARY

In accordance with the purpose(s) of the present disclosure, as embodied and broadly described herein, the disclosure, in one aspect, relates to pregnane neurosteroid analogues of 5α-pregnan-3α-ol-20-one (3α,5α-THP). Also disclosed herein are methods of making the same, pharmaceutical compositions comprising the same, and methods of treating HIV and/or neuroHIV using the same. The pharmaceutical compositions can alleviate at least one symptom associated with HIV and/or neuroHIV and can be used alone or in combination with other HIV therapies including combination antiretroviral therapy (cART).

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows HIV-1 proteins, Tat and/or gp120, dysregulate the HPA axis and promote cognitive and affective dysfunction. cART cannot target Tat or eradicate HIV in the CNS. New adjunct therapeutics are needed.

FIG. 2 shows steroidogenic compounds that are neuroprotective against HIV-1 or its toxic proteins.

FIG. 3 shows high concentrations of corticosterone reduce mitogen-stimulated proliferation of primary, mouse splenocytes. However, splenocytes cultured from Tat-expressing mice (Tat+) demonstrated glucocorticoid insensitivity at the highest concentration (5 μM) compared to non-Tat-expressing controls (Tat-; open circles), *p<0.05.

FIG. 4 shows Transgenic female mice that expressed Tat (Tat+; hatched bar), had elevated corticotropin releasing hormone (CRH) in hypothalamus compared to controls (Tat-; open bar), *p<0.05.

FIG. 5 shows that, as seen in HIV+ patients, transgenic male mice that expressed Tat (Tat+, hatched bars) had higher basal glucocorticoid than did controls (Tat-, open bars; see *). The glucocorticoid receptor antagonist, RU-486, blocks the adrenal signal, elevating corticosterone (see †); however, Tat+ males showed adrenal insufficiency (see ‡), recapitulating the clinical phenotype, p<0.05.

FIG. 6A shows Ligand structural alignment reveals structural similarities among dCa, 3α,5α-THP, and(S)-equol. FIG. 6B shows Tat in a surface representation in the zoom-out view and in a ribbon representation in the zoom-in view (top and bottom boxes). Key residues of Tat for ligand binding are shown in stick representation

FIG. 7 shows disclosed 3α, 5α-THP analogues.

FIG. 8 shows an overlay of molecular docking of dCa and compound 1 in the HIV-1 Tat (PDB: 1K5K)

FIG. 9 shows alloP or analogue 1 attenuates HIV-1BaL (1 ng/mL p24) replication over 10 days as indicated by the accumulation of p24 HIV capsid protein. Neither AlloP nor 1 impairs the efficacy of cART when co-administered.

FIGS. 10A-10B show HIV-1 Tat and/or gp120 produced necrosis of mouse primary medium spiny neurons in mixed glial co-culture following 20 h (see *). Pretreatment with 3α,5α-THP (FIG. 10A) or analogue 1 (FIG. 10B) significantly attenuated necrosis at every dose assessed (see †). The highest concentrations of either compound increased baseline cell death modestly (see \hat{ }), but were still protective, p<0.05.

FIGS. 11A-11B show transgene constructs for (FIG. 11A) gp120-tg and (FIG. 11B) Tat-tg mice.

FIG. 12A shows an overlaid structure of exemplary compound 1 and α₁-β₃ intersubunit site of GABA_{A}R (PDB ID 6153). FIG. 12B shows RMSD of trans-diol ligand (compound 1) and side chains of the binding residues in a molecular dynamics simulation.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

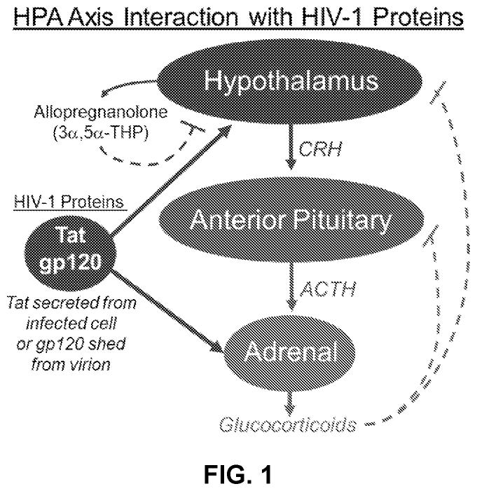

Neurosteroids, particularly allopregnanolone (also known as 5α-pregnan-3α-ol-20-one, allopregnanolone, alloP, or 3α,5α-THP), serve as non-traditional mediators of the HPA stress axis (FIG. 1). Unlike gonadally-derived steroid hormones, neurosteroids are derived de novo in CNS cells. They are rapidly synthesized in response to a range of physiological and psychological stressors and exert rapid paracrine effects to downregulate CRH and dampen the excitatory tone of the CNS to restore excitatory/inhibitory balance. This is due to their potent, allosteric activation of anion channels (largely GABAA receptors) with some neurosteroids, such as 3α,5α-THP, exerting additional effects to block cation channels (L-type Ca²⁺ channels) and other actions. Reductions in HPA-related neurosteroids have been found predict depressive symptoms in HIV-1 patients. Furthermore, Tat expression in mice has been shown to selectively activate HPA-related neurosteroids in the CNS similar to what is seen after CNS trauma. Furthermore, it has been found that 3α,5α-THP ameliorates Tat-mediated mitotoxicity, Ca²⁺ increases, and neurotoxicity in primary neural cells as well as having in vivo effects to increase anxiety-like behavior of mice. Thus, Tat activates the HPA and 3α,5α-THP synthesis, and 3α,5α-THP administration exerts anti-Tat effects.

In one aspect, the therapeutic potential of 3α,5α-THP has been sought for several disease states including epilepsy, traumatic brain injury, Alzheimer's disease, and non-Alzheimer's related dementias. Despite its promise in preclinical models, 3α,5α-THP poses a challenge to administer via routes other than intravenous administration. In rabbits or mice, the elimination half-life (t_{1/2z}) for 3α,5α-THP in brain may be ˜4 h when infused IV, but is reportedly between 12-40 min when administered subcutaneously. In people, plasma clearance may appear slower (˜4 h in women following three consecutive SC injections), but animal studies reveal rapid redistribution from the CNS and peripheral accumulation in fat tissue. In a further aspect, consistent with this, an IV formulation is FDA-approved for postpartum depression, administered under hospital care.

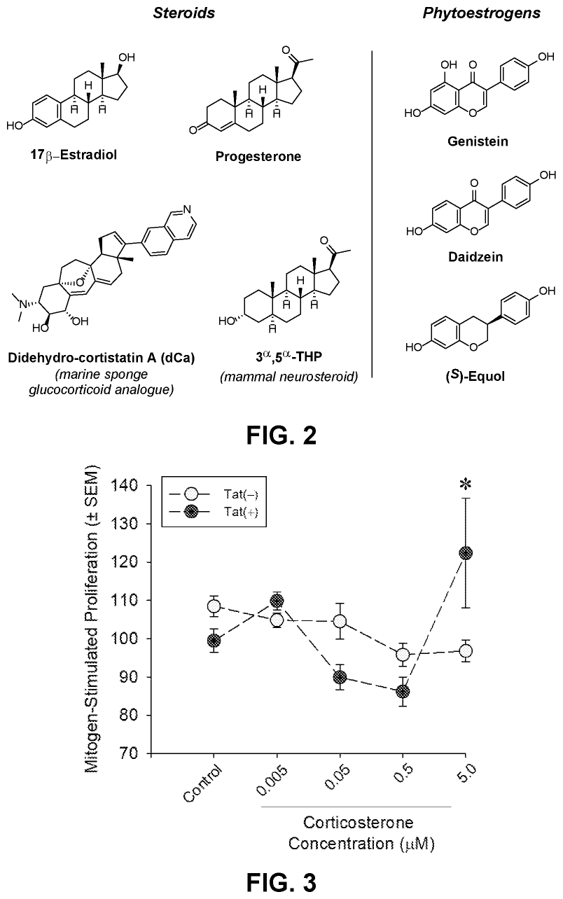

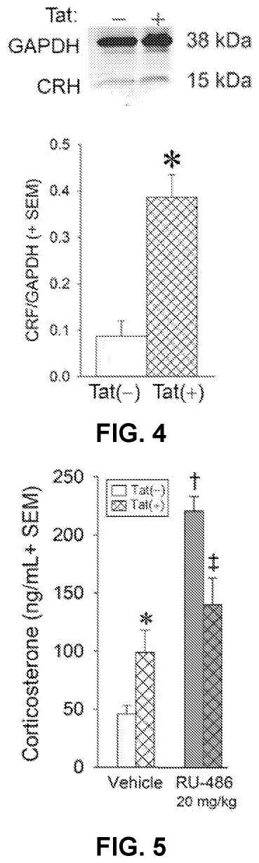

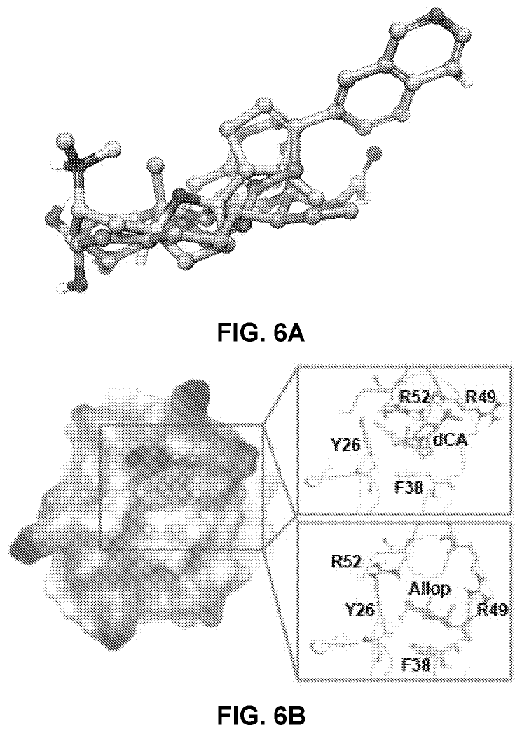

A number of studies demonstrate sex steroids, such as estradiol or progesterone (FIG. 2), to reduce HIV-1 viremia in culture and to attenuate Tat-mediated neurotoxicity. Progesterone's protective effects have been found to be due to its conversion to 3α,5α-THP (FIG. 2). Phytoestrogens, such as(S)-equol (FIG. 2), have been to exert similar properties, and an analogue of a marine sponge glucocorticoid, didehydro-cortistatin A (dCA; FIG. 2), has been well-characterized for its anti-viremic and pro-immune effects. In particular, dCA binds potently (1-2 nM affinity) and selectively with HIV-1 trans-activation response element to reduce viral transcription. A ligand structural alignment of dCa, (S)-equol, and 3α,5α-THP reveals structural similarities that may confer their anti-HIV activities (FIGS. 6A-6B). For instance, both terminal ends of the molecules contain polar elements (an oxygen or a nitrogen atom). Molecular modeling of dCa on the NMR-derived structure of Tat reveals that the nitrogen atom in the isoquinoline moiety and the two hydroxyl functional groups positioned in anti-position in the cycloheptane ring interact with the N-terminal and basic regions of Tat, respectively. Disclosed herein is a structure-based approach to create diverse 3α,5α-THP analogues with greater CNS retention and longer elimination half-lives, as well as analogues created using this approach.

In some aspects, stereospecific functionalization at the C2 position of 3α,5α-THP is particularly effective for anti-HIV-1 activity in vitro and in vivo.

In one aspect, disclosed herein is a compound including a structure of Formula I,

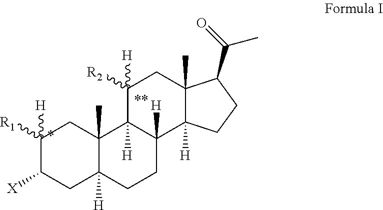

wherein R₁ and R₂ individually are H, halogen, CF₃, CH₂CF₃, OH, SH, OR₃, SR₃, substituted or unsubstituted C3-C7 aryl or heteroaryl, amino, C1-C10 alkylamino, C1-C10 dialkylamino, acyl, C1-C10 alkyl ester, or any combination thereof;
wherein R₃ is linear or branched C1-C10 alkyl, CF₃, CH₂CF₃, substituted or unsubstituted C3-C7 aryl or heteroaryl, or any combination thereof; and
wherein X is OR₄;
   wherein R₄ is H; or
wherein R₁ and X together are an oxygen atom, forming an epoxide;
provided R₁ and R₂ are not both H.

Further in this aspect, when R₁ and X are an oxygen atom, forming an epoxide, the compound can have a structure of Formula Ia:

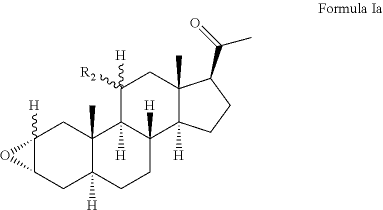

In another aspect, R₁ and R₂ individually can be selected from H, OH, dimethylamino, OCF₃, OCH₃, OCH₂CF₃, NH₂, or any combination thereof;
provided R₁ and R₂ are not both H

In still another aspect, when R₁ is not H, the structure of Formula I can have substantially R stereochemistry, substantially S stereochemistry, or a mixture thereof at the carbon atom indicated by * or the structure can have from about 5% to about 95% R stereochemistry and from about 95% to about 5% S stereochemistry at the carbon atom indicated by *.

In still another aspect, when R₁ is not H, the structure of Formula I can have substantially R stereochemistry, substantially S stereochemistry, or a mixture thereof at the carbon atom indicated by ** or the structure can have from about 5% to about 95% R stereochemistry and from about 95% to about 5% S stereochemistry at the carbon atom indicated by **.

In another aspect, the structure of Formula I can be selected from

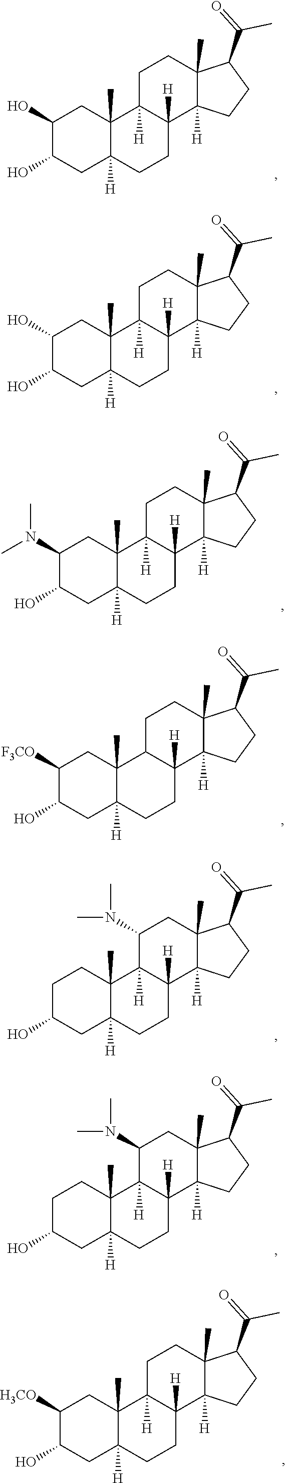

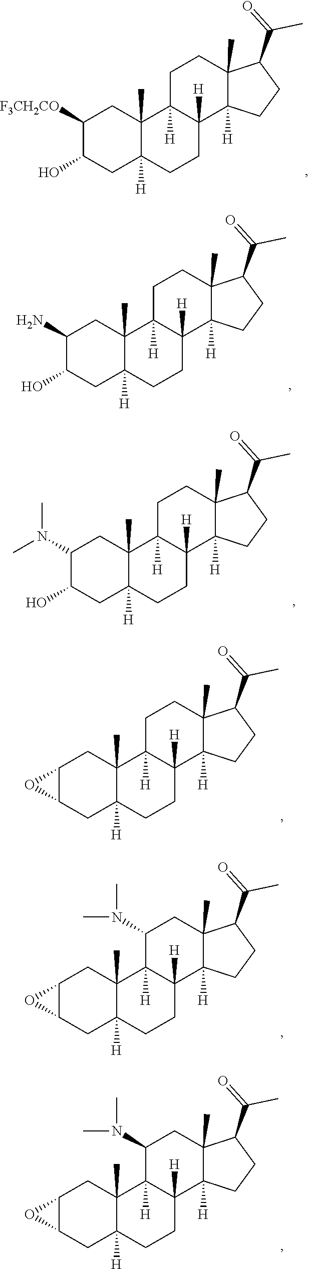

or any combination thereof.

In still another aspect, disclosed herein is a pharmaceutical composition that includes a therapeutically effective amount of a disclosed compound or a pharmaceutically acceptable salt thereof. In another aspect, disclosed herein is a method for treating HIV or neuroHIV in a subject, the method including at least the step of administering a disclosed compound or pharmaceutical composition to the subject. In one aspect, the compound or composition is administered such that from about 1 mg to about 10 mg of the compound is delivered per 1 kg of body weight of the subject, or about 1, 1.5, 2, 2.5, 3, 3.5, 4, 4.5, 5, 5.5, 6, 6.5, 7, 7.5, 8, 8.5, 9, 9.5, or about 10 mg/kg of the compound, or a combination of any of the foregoing values, or a range encompassing any of the foregoing values.

In another aspect, the compound or composition can be administered orally, intravenously, subcutaneously, or via an implanted device. In some aspects, the compound can be administered daily. In another aspect, the subject can be a human.

Further in this aspect, administering the pharmaceutical composition to a subject can reduce at least one symptom associated with HIV or neuroHIV in the subject. Still further in this aspect, the at least one symptom can be selected from a neurocognitive disorder, a neuropsychiatric disorder, a deficit in learning or memory, behavioral inhibition, affective well-being, hypothalamic-pituitary-adrenal (HPA) stress-axis dysfunction, hypothalamic-pituitary-gonadal (HPG) axis dysfunction, hypothalamic-pituitary-thyroid (HPT) axis dysfunction, central nervous system viremia, elevated glucocorticoid levels, adrenal insufficiency, gonadal hormone insufficiency, glucocorticoid insensitivity, anxiety, depression, neurotoxicity of HIV medications, or any combination thereof. In another aspect, administering the pharmaceutical composition to the subject inhibits production or activity of at least one HIV-associated protein such as, for example, trans-activator of transcription protein (Tat), envelope proteins (gp41 and/or gp120), p55, p24, p17, p6, p7, Rev, negative factor (Nef), viral protein R (Vpr), viral protein U (Vpu), virion infectivity factor (Vif), or any combination thereof.

Also disclosed herein are methods for treating HIV or neuroHIV in a subject, the methods including administering the disclosed pharmaceutical compositions to the subject. In some aspects, the methods can further include administering at least one additional HIV treatment to the subject such as, for example, combination antiretroviral therapy (cART). In a further aspect, CART and/or the at least one additional HIV treatment can include treatment with a nucleoside reverse transcriptase inhibitor (e.g. abacavir, emtricitabine, lamivudine, tenofovir disoproxil fumarate, or zidovudine), a non-nucleoside reverse transcriptase inhibitor (e.g. doravirine, efavirenz, etravirine, nevirapine, or rilpivirine), a protease inhibitor (atazanavir, darunavir, fosamprenavir, ritonavir, or tipranavir), a fusion inhibitor (e.g. enfuvirtide), a CCR5 antagonist (e.g. maraviroc), an integrase strand transfer inhibitor (e.g. cabotegravir, dolutegravir, or raltegravir), an attachment inhibitor (e.g. fostemsavir), a post-attachment inhibitor (e.g. ibalizumab-uiyk), a pharmacokinetic enhancer (e.g. cobicistat), or any combination thereof. In any of these aspects, the disclosed pharmaceutical compositions and the at least one additional HIV treatment can act synergistically to reduce HIV symptoms, replication, transcription, and the like. Further in this aspect, synergistic action can enable the use of lower doses of both medications in the subject, thereby reducing systemic side effects and increasing patient compliance with treatment. In another aspect, administering the compound of Formula I reduces cytotoxicity of cART relative to administration of cART without performing the method.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a substituent,” “a stressor,” or “an analogue,” include, but are not limited to, mixtures or combinations of two or more such substituents, stressors, or analogues, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a drug refers to an amount that is sufficient to achieve the desired improvement in the property modulated by the formulation component, e.g. achieving the desired level of regulation of neuroHIV or other neurodegenerative disease symptoms. The specific level in terms of wt % in a composition required as an effective amount will depend upon a variety of factors including the nature of any other therapies being administered, patient's viral load, method of administration, and the like.

As used herein, the terms “optional” or “optionally” means that the subsequently described event or circumstance can or cannot occur, and that the description includes instances where said event or circumstance occurs and instances where it does not.

Unless otherwise specified, pressures referred to herein are based on atmospheric pressure (i.e. one atmosphere).

### Chemical Groups and Substituents

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic.

The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

The terms “alkoxy” and “alkoxyl” as used herein to refer to an alkyl or cycloalkyl group bonded through an ether linkage; that is, an “alkoxy” group can be defined as -OA¹ where A¹ is alkyl or cycloalkyl as defined above. “Alkoxy” also includes polymers of alkoxy groups as just described; that is, an alkoxy can be a polyether such as -OA¹-OA² or -OA¹-(OA²)ₐ-OA³, where “a” is an integer of from 1 to 200 and A¹, A², and A³ are alkyl and/or cycloalkyl groups.

The term “aldehyde” as used herein is represented by the formula —C(O) H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH (-alkyl) and-N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O) A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA2, where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “nitro” as used herein is represented by the formula —NO₂.

The term “nitrile” or “cyano” as used herein is represented by the formula —CN. The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯}C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯}N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O) SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯}S(O)₂NR^{◯}₂; —N(R^{◯})S(O)₂R^{◯}; —N(OR^{◯})R^{◯}; —C(NH)NR^{◯}₂; P(O)₂R^{◯}; —P(O)R^{◯}₂; —OP(O)R^{◯}₂; —OP(O)(OR)₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O) SR^{●}—(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and =S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R′ is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R′ is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include-R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and(S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or(S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine, and chlorine, such as ²H, ³H, ¹³C, ¹⁴C, ¹⁵N, ¹⁸O, ¹⁷O, ³⁵S, ¹⁸F, and ³⁶Cl, respectively.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as HIV and/or neuroHIV. The effect can be therapeutic in terms of a partial or complete cure of a disease, condition, symptom, or adverse effect attributed to the disease, disorder, or condition. The term “treatment” as used herein can include any treatment of HIV and/or neuroHIV or another neurodegenerative disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

### Pharmaceutical Compositions

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets, or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentaerythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate, and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate, and cellulose acetate phthalate.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The following list of exemplary aspects supports and is supported by the disclosure provided herein.

Aspect 1. A compound comprising a structure of Formula I

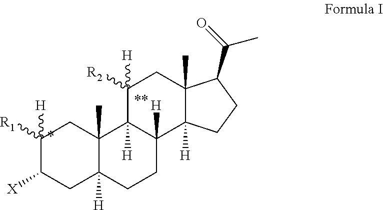

wherein R₁ and R₂ individually are H, halogen, CF₃, CH₂CF₃, OH, SH, OR₃, SR₃, substituted or unsubstituted C3-C7 aryl or heteroaryl, amino, C1-C10 alkylamino, C1-C10 dialkylamino, acyl, C1-C10 alkyl ester, or any combination thereof;
wherein R₃ is linear or branched C1-C10 alkyl, CF₃, CH₂CF₃, substituted or unsubstituted C3-C7 aryl or heteroaryl, or any combination thereof; and
wherein X is OR₄;
   wherein R₄ is H; or
wherein R₁ and X together are an oxygen atom, forming an epoxide;
provided R₁ and R₂ are not both H.

Aspect 2. The compound of aspect 1, wherein R₁ and R₂ individually are selected from H, OH, dimethylamino, OCF₃, OCH₃, OCH₂CF₃, NH₂, or any combination thereof;
provided R₁ and R₂ are not both H.

Aspect 3. The compound of aspect 1 or 2, wherein when R₁ is not H, the structure of Formula I has substantially R stereochemistry, substantially S stereochemistry, or a mixture thereof at the carbon atom indicated by *.

Aspect 4. The compound of aspect 1 or 2, wherein when R₁ is not H, the carbon atom indicated by * has from about 5% to about 95% R stereochemistry and from about 95% to about 5% S stereochemistry.

Aspect 5. The compound of any one of aspects 1-4, wherein when R₂ is not H, the structure of Formula I has substantially R stereochemistry, substantially S stereochemistry, or a mixture thereof at the carbon atom indicated by **.

Aspect 6. The compound of any one of aspects 1-4, wherein when R₂ is not H, the carbon atom indicated by ** has from about 5% to about 95% R stereochemistry and from about 95% to about 5% S stereochemistry.

Aspect 7. The compound of any one of aspects 1-6, wherein the structure of Formula I is selected from

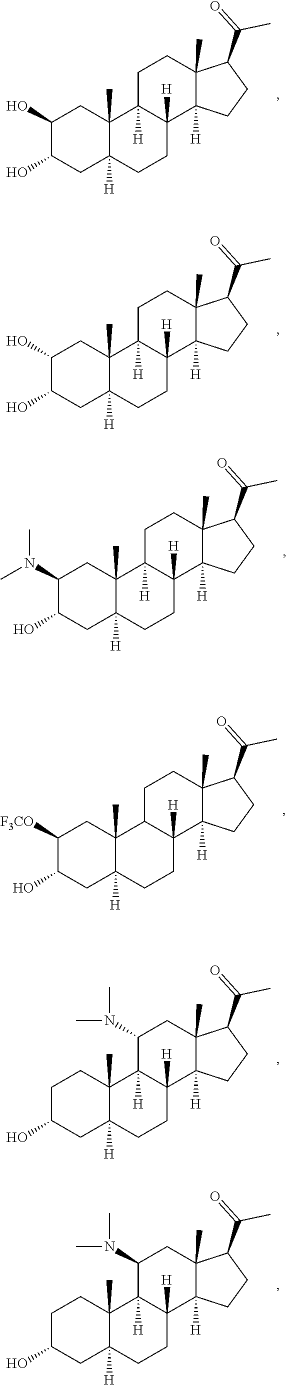

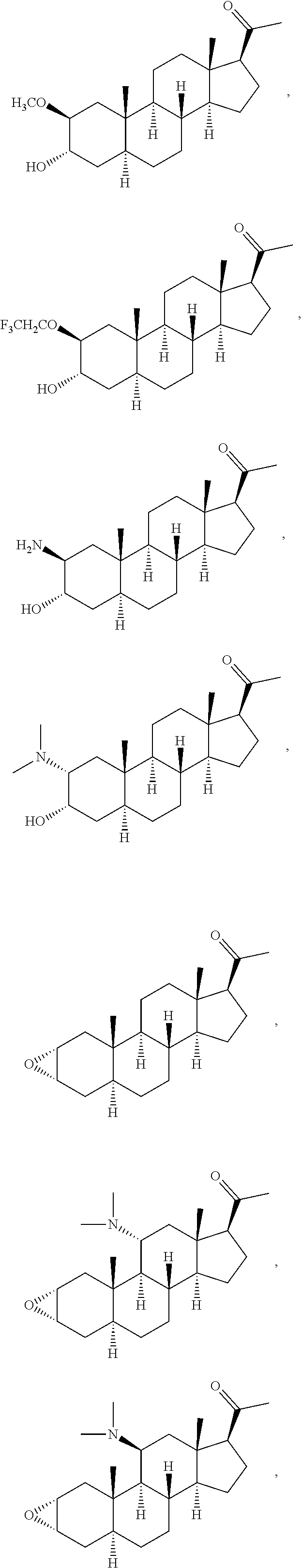

or any combination thereof.

Aspect 8. A pharmaceutical composition comprising a therapeutically effective amount of the compound of any one of aspects 1-7 or a pharmaceutically acceptable salt thereof.

Aspect 9. A method for treating HIV or neuroHIV in a subject, the method comprising administering the compound of any one of aspects 1-7 or the composition of aspect 8 to the subject.

Aspect 10. The method of aspect 9, wherein administering the compound or composition comprises administering from about 1 mg to about 100 mg of the compound per kg of body weight of the subject.

Aspect 11. The method of aspect 9 or 10, wherein the compound or composition is administered orally, intravenously, subcutaneously, or via an implanted device.

Aspect 12. The method of any one of aspects 9-11, wherein the compound is administered daily.

Aspect 13. The method of any one of aspects 9-12 wherein the subject is a human.

Aspect 14. The method of any one of aspects 9-13, wherein administering the pharmaceutical composition to the subject reduces at least one symptom associated with HIV or neuroHIV in the subject.

Aspect 15. The method of aspect 14, wherein the at least one symptom comprises a neurocognitive disorder, a neuropsychiatric disorder, a deficit in learning or memory, behavioral inhibition, affective well-being, hypothalamic-pituitary-adrenal (HPA) stress-axis dysfunction, hypothalamic-pituitary-gonadal (HPG) axis dysfunction, hypothalamic-pituitary-thyroid (HPT) axis dysfunction, central nervous system viremia, elevated glucocorticoid levels, adrenal insufficiency, gonadal hormone insufficiency, glucocorticoid insensitivity, anxiety, depression, neurotoxicity of HIV medications, or any combination thereof.

Aspect 16. The method of any one of aspects 9-15, wherein administering the pharmaceutical composition to the subject inhibits production or activity of at least one HIV-associated protein.

Aspect 17. The method of aspect 16, wherein the at least one HIV-associated protein comprises trans-activator of transcription protein (Tat), envelope proteins gp41, envelope protein gp120, p55, p24, p17, p6, p7, Rev, negative factor (Nef), viral protein R (Vpr), viral protein U (Vpu), virion infectivity factor (Vif), or any combination thereof.

Aspect 18. The method of any one of aspects 9-17, further comprising administering at least one additional HIV treatment to the subject.

Aspect 19. The method of aspect 18, wherein the at least one additional HIV treatment comprises combination antiretroviral therapy (cART).

Aspect 20. The method of aspect 19, wherein the compound of Formula I reduces cytotoxicity of cART relative to administration of cART without performing the method.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Synthesis of 3α,5α-THP Analogues

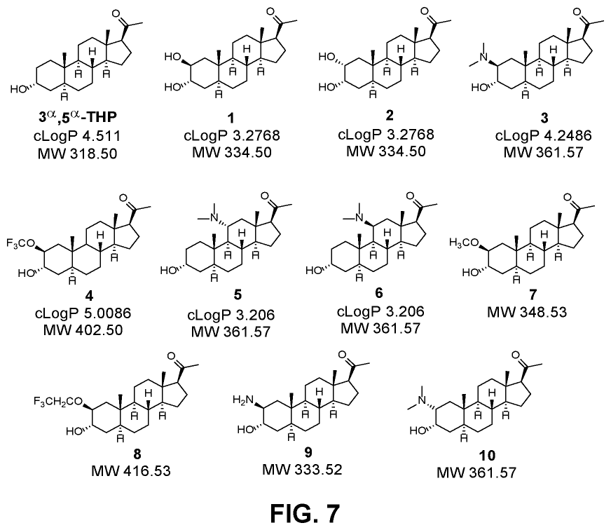

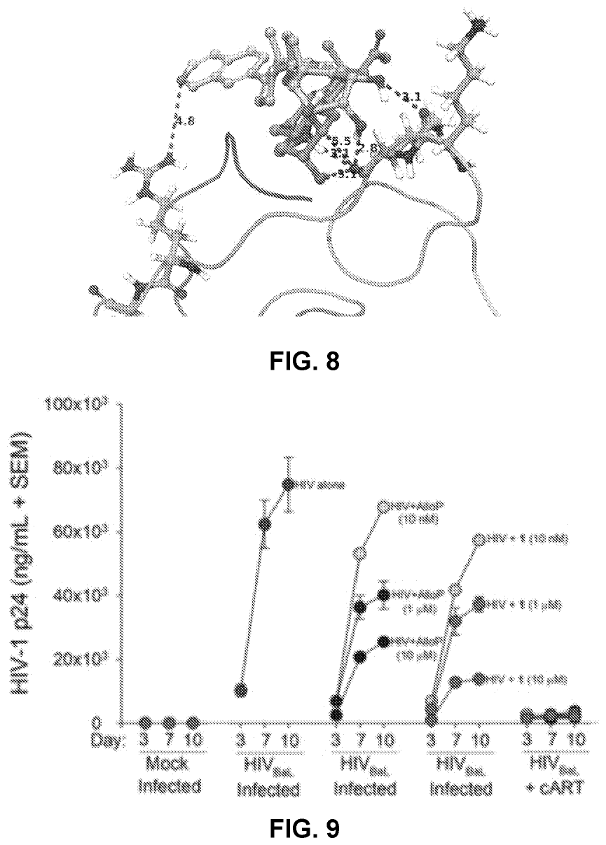

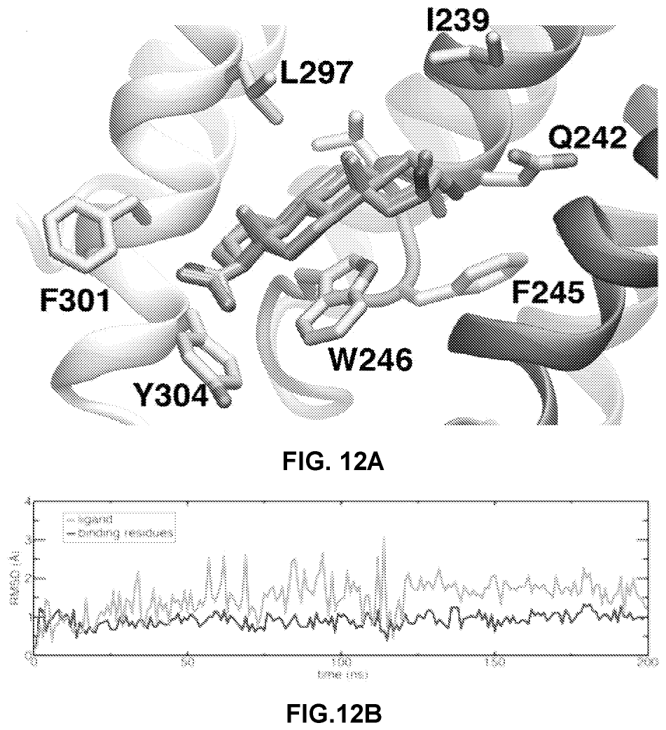

Key features of dCA, such as trans-diol (compound 1) and trans-hydroxy-dimethylamine (compound 3) groups, were built into the structure of 3α,5α-THP and docked these compounds into the binding pocket of HIV-1 Tat. This computational modeling has led to a diverse library of promising compounds (1-6, FIG. 7) with excellent docking scores and similar conformations and interactions to the basic regions of Tat, the same regions that dCa binds. As an example, FIG. 8 shows the overlay of the docked poses of dCa and compound 1 with Tat, indicating similar interactions and binding modes. Different directions of the physiochemical properties were also considered, such as lipophilicity, molecular weights, hydrogen bonding, charges, and polar surface areas to ensure a diverse library while maintaining their PK/PD and BBB-permeability. In support, compounds 1-3 have cLogP values in the range of 3.2-4.2 vs. 4.5 of 3α,5α-THP, indicating that they are slightly less lipophilic than 3α,5α-THP, which may improve solubility and reduce rapid redistribution to fat cells. Compound 4 has a c Log P value of 5.5, slightly more lipophilic than 3α,5α-THP, potentially improving CNS accumulation. Compound 4 also possesses a trifluoromethyl group, and fluorinated steroids are found to have improved stability and activity. Compound 1 is equipotent to 3α,5α-THP in ameliorating Tat-neurotoxicity. Further results of molecular modeling and molecular dynamics simulations are presented in FIGS. 12A-12B.

Given that compound 1 was developed using structure-based drug design coupled with computational modeling and produced a 3α,5α-THP analogue with equipotent neuroprotection against Tat, compounds 2-6 were expected to exert a similar or greater neuroprotective profile.

The synthesis of compound 1 was begun from commercially-available compound 7 (Scheme 1). The hydroxyl group of 7 was tosylated to make it a better leaving group, which was then subjected to an elimination reaction that produced a mixture of compounds 9 and 10. This mixture was subjected to an epoxidation reaction without separation. The epoxide 11 was purified and hydroxylated to give compound 1 with the trans-diol group at the 2,3 positions. All 4 steps of this synthetic route resulted in good yields.

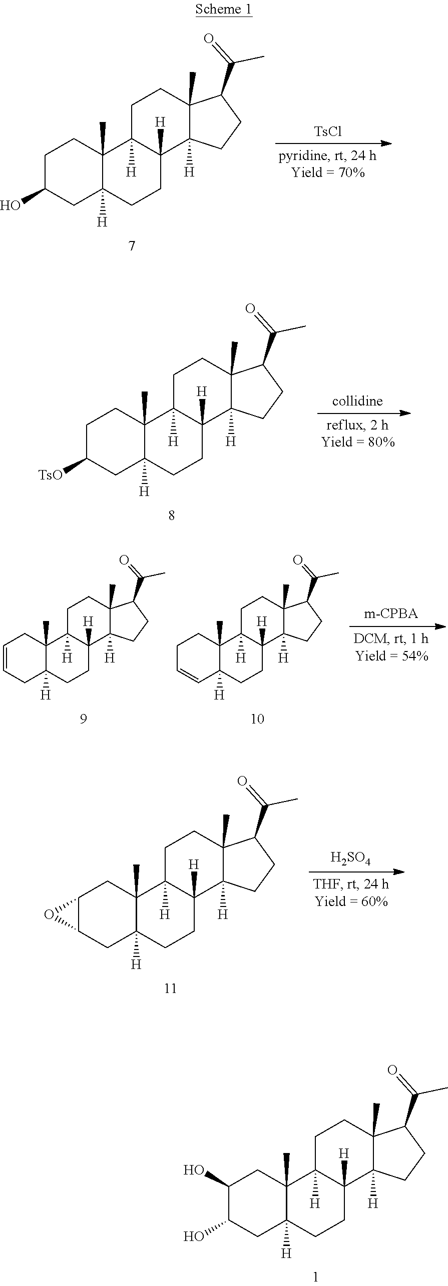

Compounds 2-6 were synthesized and tested for their anti-HIV-1 capacity in vitro. To synthesize compound 2, the same synthetic route for compound 1 was followed, starting from 7, up until the mixture of compounds 9 and 10 is achieved. The mixture was subjected to an oxidation reaction using osmium tetroxide; the products were separated via column chromatography and preparative HPLC to give compound 2 with a cis-diol group at the 2,3 positions (Scheme 2).

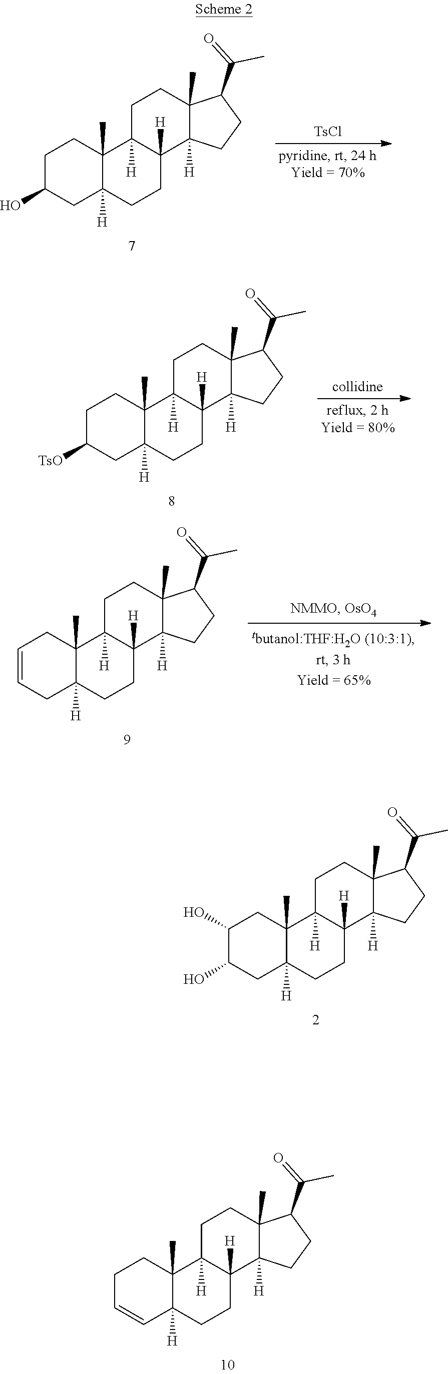

To synthesize compound 3, compound 11 was used as a starting material (synthesized from 7, as above). An aminolysis of the epoxide led to 3 (Scheme 3).

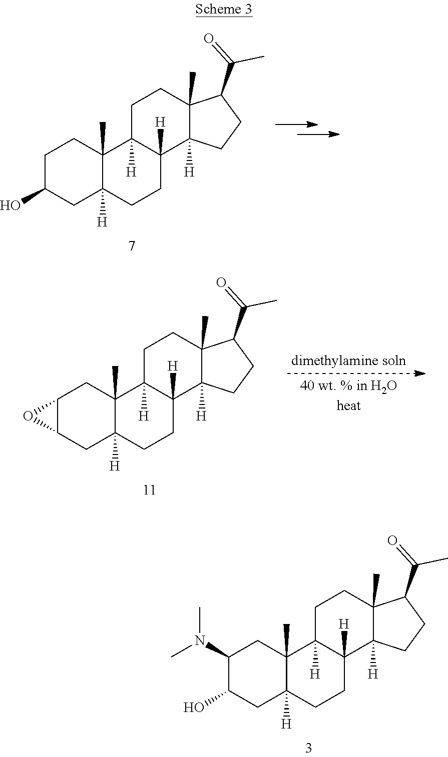

The synthesis of compound 4 also started from 11. The hydroxyl group at position 3 was protected, whereas the hydroxyl group at position 2 was converted to S-methyl xanthate to synthesize compound 13. After that, 13 was reacted with hydrogen fluoride to incorporate the trifluoromethoxy group at position 2 to give compound 14. Deprotection of 14 with trifluoroacetic acid led to compound 4 (Scheme 4).

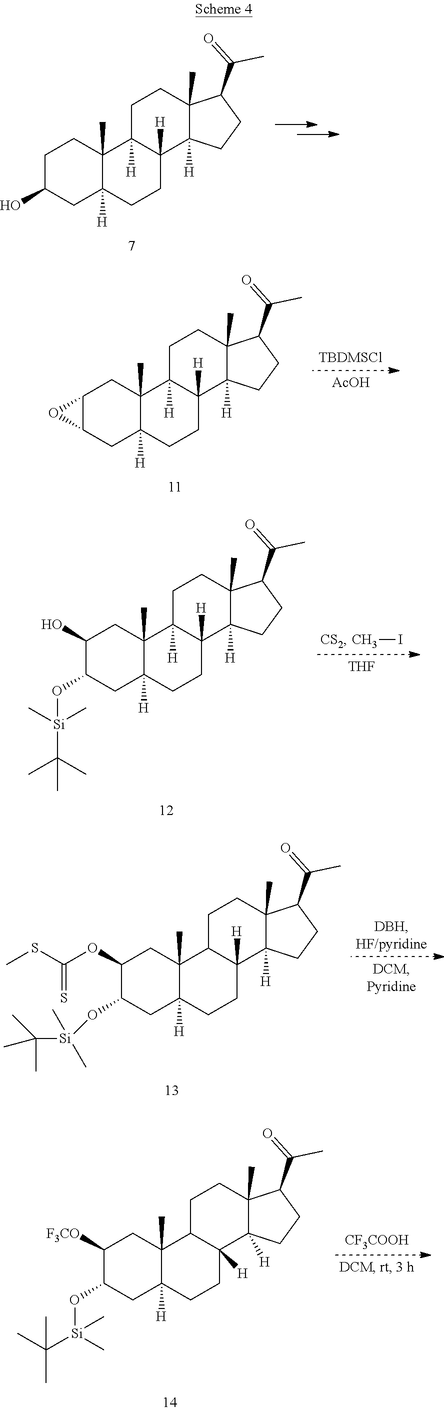

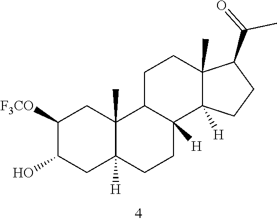 [SMILES: CC(=O)[C@H]1CC[C@H]2[C@@H]3CC[C@H]4C[C@H](O)[C@@H](OC(F)(F)F)C[C@]4(C)C3CC[C@]12C]

Compound 5 was synthesized in 5 steps from commercially available compound 15. The acetyl group of 15 was protected using ethylene glycol to get compound 16. The carbonyl group at position 3 of 16 was reduced to a hydroxyl group to result in compound 17, which was further reacted with ammonium hydroxylamine and further reduced to give the 11-α amine on ring C. Finally, the hydrogens on the amine were substituted with methyl groups to produce compound 5 (Scheme 5).

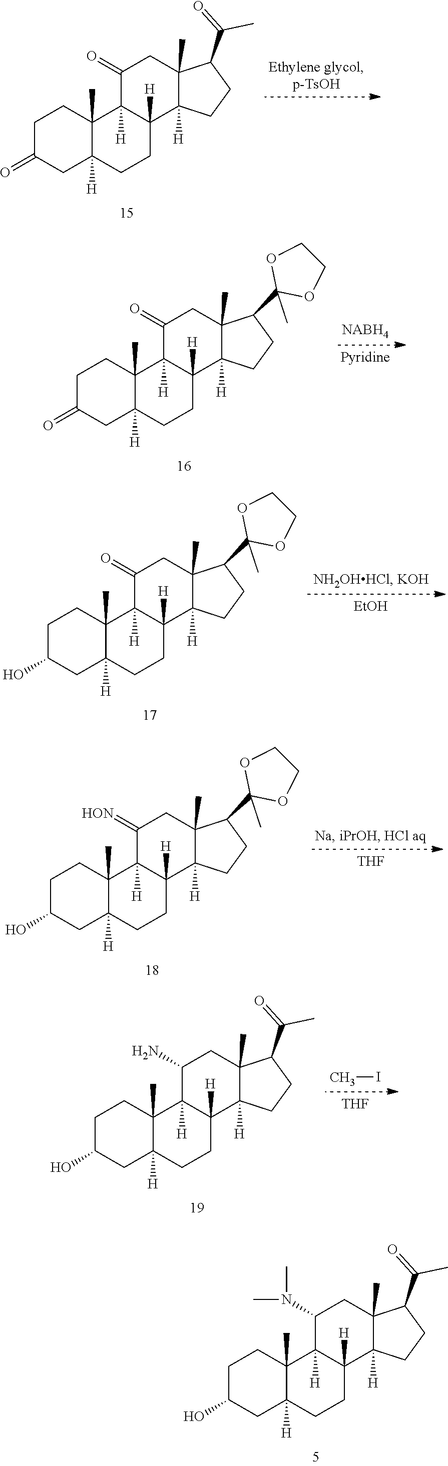

Compound 6 was synthesized starting with compound 18 (synthesized from 15 as above). Compound 18 was reduced to get the 11-β amine on ring C. A reaction between methyl iodide and compound 20 produced compound 6 (Scheme 6).

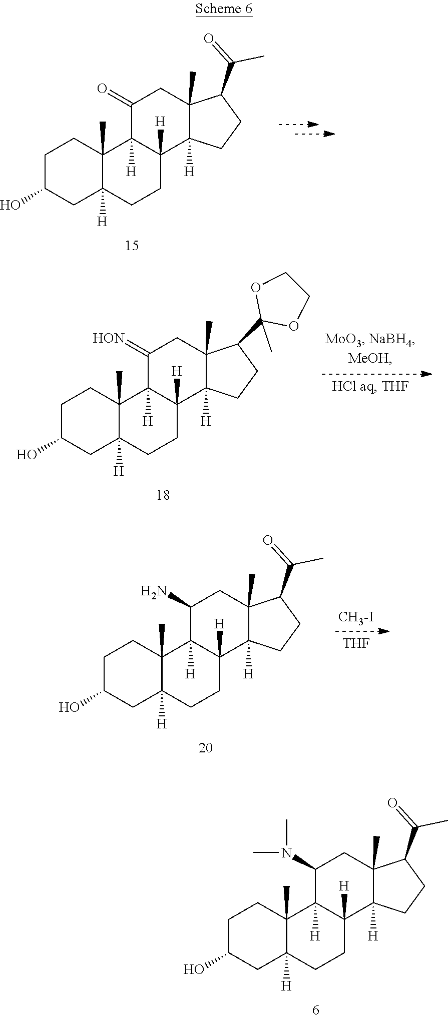

Major issues were not expected in the synthesis of compounds 2-6, but potential complications were considered. The aminolysis of epoxide 11 in water to give 3 could, under some circumstances, result in a low yield. Addition of a Lewis acid catalyst, such as Zn (ClO₄)₂, Cs₂CO₃, LiClO₄, or the like, can help increase the yield of this aminolysis.

Different reaction conditions are proposed to selectively produce 19 and 20 from 18 to get the amine on position 11 on ring C. However, a mixture of both 19 and 20 is likely to be produced. In this case, 19 and 20 can be separated and purified prior to continuing the synthesis of 5 and 6, respectively. Similarly, the final reactions to produce 2 and 3, as well as 12, produce mixtures. Column chromatography and preparative HPLC can be used for these separations and purifications.

### Example 2: In Vitro Screening of 3α,5α-THP Analogues for Anti-HIV-1 Capacity

3α,5α-THP and one or more analogues significantly reduce HIV-1 viremia, cytokine production, and cytotoxicity, and do not exert cytotoxic interactions with cART. Primary human microglia (ScienCell, CA or an alternate source: CellProgen, CA) were cultured and mock-infected or infected with HIV-1BaL (100 ng/ml HIV p24; 15 days; NIH AIDS Reagent Program or Immune Tech. Corp.) as described previously.

Assessment for viremia: Media, compounds 1-6, or 3α,5α-THP was assessed in a dose-response curve (0.1, 0.3, 1, 3.2, 10, 32, 100, 320, 1×10³, 10×10³, 100×10³ nM) for their anti-viremic capacity (FIG. 9). To monitor infection, samples are taken every 72 h to measure p24 or proviral DNA129-130. Infection was confirmed post-hoc via immunocytochemical imaging of HIV-1 negative regulatory factor (Nef). As needed, TZM-bl and Jurkat reporter cells were also used to determine viral proliferation.

Cytotoxicity (ICC): Cell death in cultures was determined by co-labeling with Hoechst nuclear stain and propidium iodide. In each sample, at least 100 cells were counted with two technical replicates and 8 independent experiments representing each condition.

Cytokine ELISA: IL-1B, IL-6, TNF-α, CCL2, and CCL5 were assessed via ELISA as described previously.

Interactions with cART: Efficacious concentrations were re-assessed in separate mock- or HIV-1_{BaL}-infected cultures in the presence or absence of Triumeq® [Abacavir (ABC; 10 μM)/dolutegravir (DTG; 1 nM)/lamivudine (3TC; 30 μM) dosing derived from FDA-approved insert] to assess interactions with viremic control, cytotoxicity, and cytokine production.

Additional Experiments: If efficacious analogues are devoid of cytotoxic interactions with CART, an additional experiment can be conducted wherein cART concentrations were reduced to assess the capacity for analogues to bolster the anti-viremic effects of a lower concentration of CART. This is desirable to reduce acute and long-term side effects associated with cART, particularly benefitting patients with early-life infection.

Infection and analogue interactions with cART may also be confirmed via the use of TZM-bl or CCR5-expressing Jurkat HIV-1 LTR-reporter cell lines that are also currently used for screening.

### Example 3: Pharmacokinetics of 3α,5α-THP Analogues in Mouse Models

Lead compounds should exert greater CNS accumulation with longer t_{1/2z} than 3α,5α-THP, and HIV-1 Tat or gp120 are expected to increase anxiety- and depression-like behavior, perturb spatial cognitive performance, and dysregulate the HPA axis. Furthermore, 3α,5α-THP or the lead analogue can attenuate these effects and is devoid of interactions with cART.

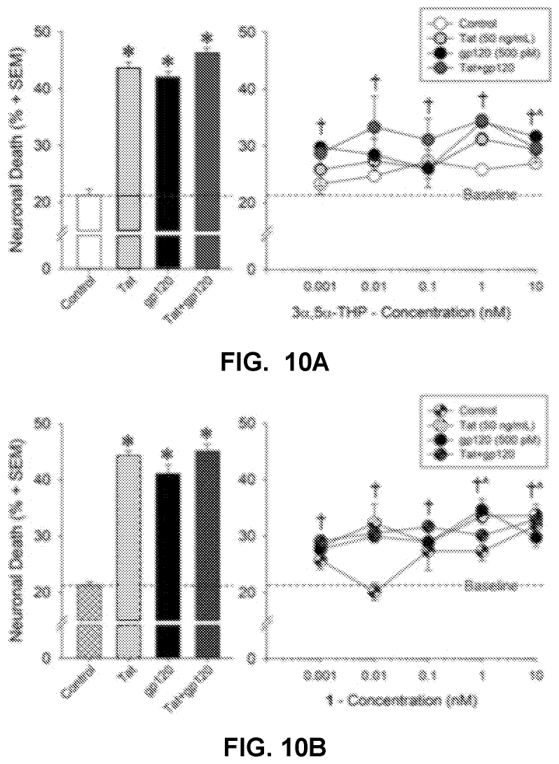

The trans-diol analogue of 3α, 5α-THP, 5α-pregnan-2β,3α-diol-20-one (compound 1) was synthesized. Mixed glia (derived from 0-1 day old mouse pups) and primary medium spiny neurons (from the striatum of E17 pups) were co-cultured as previously described. Co-cultures were pretreated with a concentration-response curve (0.001-10×10³ nM) of 3α,5α-THP or the analogue, 1, and exposed to HIV-1 Tat and/or gp120 for 20 h. Prior time-lapse experiments have identified this time-point as the earliest time at which 3α,5α-THP protection is evident. Each treatment group consisted of 3 independent observations (with 2 technical replicates each). Necrotic cells were identified via propidium iodide labeling. Either Tat, gp120, or the combination of both, significantly increased neuron cell death, as expected (FIGS. 10A-10B). Any concentration of 3α,5α-THP (FIG. 10A) or analogue 1 (FIG. 10B) significantly attenuated Tat and/or gp120-mediated neurotoxicity. Notably, cell death was increased by the highest concentration of 3α,5α-THP (5.7% increase at 10 nM) and 1 (8.8% and 10.5% increases at 1 and 10 nM, respectively).

Leads are defined as compounds that significantly reduce viremia and cytotoxicity without cytotoxic interactions with cART. Compounds do not need to reduce cytokines or bolster cART potency to be considered leads.

C57BL/6J mice were administered 3α,5α-THP or lead compounds IV or SC. 3α,5α-THP dosing were administered at previously safe and effective concentrations (1 mg/kg, IV or 10 mg/kg, SC); analogue dosing was calculated from in vitro potency to 3α,5α-THP. Brain and plasma was collected from separate groups of mice at 15 min, 30 min, 1 h, 4 h, and 24 h post-administration. 3α,5α-THP or analogue content was assessed via GC/MS. Maximum plasma concentration (Cₘₐₓ), latency to Cₘₐₓ (Tₘₐₓ), t_{1/2z}, and elimination constant (k) was calculated.

### Example 4: Pro-Affective/Pro-Cognitive In Vivo Capacity of 3α,5α-THP Analogues in Mouse Models

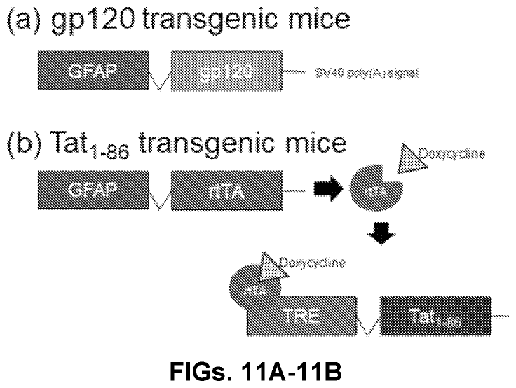

Anti-Tat/gp120 Potency/Efficacy: The single best lead compound and/or 3α,5α-THP was assessed. Tat-tg and gp120-tg mice express transgenes under regulation of a GFAP promotor (FIGS. 11A-11B) relegating the vast majority of expression to the CNS. In Tat-tg mice, expression is induced via doxycycline administration. Expression is constitutive in gp120-tg mice (C57BL/6J-gp120ADA or -gp120IIIB infused controls are also proposed).

Surgery and Treatment: Mice were implanted with osmotic minipumps as previously described containing placebo or cART, concurrent with the lead compound or 3α,5α-THP. Triumeq® (ViiV Healthcare) is purchased from the UMMC Pharmacy and dosing for mice is calculated via allometric scaling and administered via osmotic minipump (Alzet, CA).

Behavioral Testing: Five days after implantation, mice were assessed for spontaneous motor and anxiety-like behavior in an open field (day 6), depression-like behavior in the tail suspension test (day 8), and spatial cognitive performance in a Barnes maze (days 10-14).

Tissue Analyses: Minipumps delivered drug for 15 days. On day 14, brain, abdominal muscle and adipose tissue, and plasma was collected. Brains were mid-sagittally split with one hemisphere of brain, muscle, adipose tissue, and some plasma assessed for 3α,5α-THP and analogue content via GC/MS. Remaining brain was assessed for CRH in the hippocampus, striatum, midbrain, frontal cortex, and hypothalamus and remaining plasma was assessed for corticosterone via ELISA.

Additional Experiments: Dosing may need to be adjusted when transitioning to in vivo work. Dosing or minipump size (if longer administration is needed) can be increased.

Unlike the conditionally-inducible Tat-tg mice, the transgene in gp120-tg mice is constitutively expressed. C57BL/6J or Tat-tg mice can be i.t.-infused with R₅-tropic gp120ADA (ImmunoDx, MA) in order to assess the effects of a more acute exposure.

A group of females tested on the diestrous phase of their estrous cycle may also be tested, which would allow assessment of hormonal variations on current endpoints.

tat transgene expression is regulated by a reverse tetracycline-controlled transactivator (rtTA) and is notably “leaky.” Some low-level Tat is always expressed in Tat (+) mice. In addition to Tat (−) controls, some un-induced Tat (+) mice were included as an additional negative control to offset this.

Surgical intervention via implanted minipumps may alter HPA responding, irrespective of treatments. Some sham-surgery controls were included on each measure.

### Example 5: Statistical Analyses and Reproducibility

Experimental design was established using statistical power and sample size estimation with β set at 0.8 and a at 0.05. Based on prior studies, the baseline means and variance of all measures can be estimated. Power analyses (performed with G*Power Software version 3.1.9.2., Heinrich-Heine-Universität Düsseldorf) revealed in vitro studies to require n=8/group. In vivo pharmacokinetic studies have been estimated based on published work with 3α,5α-THP and require n=5-8/group. Behavioral studies are based on previous work and require n=10-12/group. Controls are included in every assay as described. As females were used on the day of proestrous, some atypically cycling females were excluded. Given these considerations, female group sizes were inflated 10%.

Statistical Analyses: All datasets were assessed for normality of distribution via the Kolmogorov-Smirnov test and equality of variance via Bartlett's test (or Levene's test for non-normal distributions). All experimental group designs are fully-factorial. Repeated measures experiments (e.g. repeated p24 sampling) were assessed via repeated measures ANOVAs (assuming normality) or Friedman's test (if non-normal). Pharmacological dose-response data was also log-transformed and assessed via non-linear interpolation to determine EC₅₀, Eₘₐₓ, and hillslope. Non-repeated endpoints were assessed via 1-to-3-way ANOVAs. Main effects were delineated by Tukey's HSD post-hocs (all pair-wise comparisons considered). Interactions were delineated via simple main effects and main effect contrasts with a corrected for family-wise error.

### Example 6: Materials and Methods

All chemicals were obtained from Sigma-Aldrich or Fisher Scientific and used as received unless specified. All syntheses were conducted with anhydrous conditions under an atmosphere of argon, using flame-dried glassware and employing standard techniques for handling air-sensitive materials unless otherwise noted. All solvents were distilled and stored under an argon or nitrogen atmosphere before use. ¹H NMR and ¹³C NMR spectra were recorded on a Bruker-400 and or a Bruker-500 spectrometer using CDCl₃, MeOD, or D₂O as the solvent. Chemical shifts (δ) were recorded in parts per million and referenced to CDCl₃ (7.26 ppm for 1H NMR and 77.16 ppm for 13C NMR), MeOD (3.31 ppm for 1H NMR and 49.00 ppm for 13C NMR), or D₂O (4.79 ppm for 1H NMR). ¹⁹F NMR spectra were recorded on a Bruker-400 spectrometer. Coupling constants (J) are in Hz. The following abbreviations were used to designate the multiplicities: s=singlet, d=doublet, t=triplet, q=quartet, quint=quintuplet, m=multiplet, br=broad. Melting points were measured using an OptiMelt automated melting point system. LC-MS were measured using an ACQUITY-Waters micromass (ESCi) system. High-resolution mass spectra (HRMS) were measured using a Synapt Q-TOF ESI-MS.

### Synthetic Procedures

3β-{[(4-methylphenyl) sulfonyl]oxy}-5α-pregnan-20-one (8): To a solution of 3B-hydroxy-5α-pregnan-20-one (1 g, 3.14 mmol) dissolved in pyridine (13 mL), (4-methylphenyl) sulfonyl chloride (2.4 g, 12.57 mmol) was added and stirred for 4 h at room temperature before pouring into water (30 mL). The solid precipitate was filtered, washed with water, and dissolved in dichloromethane. The organic layer was dried with Na₂SO₄ and the solvent was evaporated. Then crude extract was subjected to silica gel flash chromatography (hexane:EtOAc, 10:1). Concentration of the appropriate fractions in vacuo afforded 3β-{[(4-methylphenyl) sulfonyl]oxy}-5α-pregnan-20-one (3, 1.21 g, 80%). ¹H NMR (400 MHZ, CDCl₃) δ 7.81-7.73 (m, 2H), 7.35-7.26 (m, 2H), 4.40 (tt, J=10.8, 5.2 Hz, 1H), 2.42 (s, 4H), 2.08 (s, 4H), 1.96 (dt, J=11.7, 3.2 Hz, 1H), 1.87-0.99 (m, 17H), 0.98-0.77 (m, 2H), 0.76 (s, 3H), 0.68-0.54 (m, 4H). ¹³C NMR (101 MHZ, CDCl₃) δ 209.46 (s), 144.35 (s), 134.73 (s), 129.73 (s), 127.54 (s), 82.30 (s), 76.81 (s), 63.70 (s), 56.50 (s), 53.89 (s), 44.72 (s), 44.14 (s), 38.90 (s), 36.74 (s), 35.27 (d, J=10.7 Hz), 34.78 (s), 31.77 (s), 31.49 (s), 28.31 (d, J=5.9 Hz), 24.35 (s), 22.76 (s), 21.62 (s), 21.15 (s), 13.41 (s), 12.09 (s).

2,3-ene-5α-pregnan-20-one (9): 3β-{[(4-methylphenyl) sulfonyl]oxy}-5α-pregnan-20-one (3, 1.21 g, 2.56 mmol) was dissolved in collidine (14 mL) and heated at reflux for 1 h. Reaction was quenched with water (100 mL) containing hydrochloric acid (10 mL). The solid precipitate was filtered, washed with water, and dissolved with dichloromethane. The organic layer was extracted with water, dried with Na₂SO₄, evaporated under reduced pressure. Then crude extract was subjected to silica gel flash chromatography (hexane:EtOAc, 10:1). Concentration of the appropriate fractions in vacuo afforded 2,3-ene-5α-pregnan-20-one and its Δ3 isomer. (4, 615 mg, 80%). ¹H NMR (400 MHZ, CDCl₃) δ 5.65-5.49 (m, 2H), 2.53 (td, J=8.9, 2.0 Hz, 1H), 2.12 (s, 3H), 0.76 (s, 3H), 0.62 (d, J=1.7 Hz, 3H). 13C NMR (101 MHZ, CDCl₃) δ 125.81 (d, J=11.2 Hz), 63.86 (s), 56.71 (s), 53.94 (s), 44.17 (s), 41.43 (s), 39.76 (s), 39.10 (s), 35.59 (s), 34.64 (s), 31.77 (s), 31.53 (d, J=1.9 Hz), 30.26 (s), 28.63 (s), 24.40 (d, J=1.8 Hz), 22.79 (d, J=1.7 Hz), 21.01 (d, J=14.5 Hz), 13.38 (s), 11.69 (s).

2,3-epoxy-5α-pregnan-20-one (11): To a solution of 2,3-ene-5α-pregnan-20-one and its 43 isomer (0.5 g, 1.66 mmol) dissolved in dichloromethane (4 mL) was added a solution of 3-chloroperbenzoic acid (0.6 g) in dichloromethane (4 mL) at 0° C. The reaction was stirred below 25° C. for 1.5 h. Then reaction was quenched with potassium hydrogen carbonate (0.4 g). Water (5 mL) was added and the organic phase was washed sequentially with water, sodium thiosulfate solution, and water and dried over anhydrous sodium sulfate. The solvent was evaporated under reduced pressure and crude extract was subjected to flash column chromatography (n-hexane:EtOAc , 3:1) to give 2,3-epoxy-5α-pregnan-20-one (5, 262 mg, 50%). 1H NMR (400 MHZ, CDCl₃) δ 3.21-3.09 (m, 2H), 2.53 (t, J=9.0 Hz, 1H), 2.24-2.07 (m, 4H), 2.09-1.82 (m, 3H), 1.73-1.04 (m, 13H), 0.94-0.80 (m, 1H), 0.82-0.61 (m, 4H), 0.60 (s, 3H). 13C NMR (101 MHZ, CDCl₃) δ 209.69 (s), 63.76 (s), 56.49 (s), 53.56 (s), 52.41 (s), 51.00 (s), 44.06 (s), 38.94 (s), 38.27 (s), 36.21 (s), 35.63 (s), 33.65 (s), 31.59 (d, J=1.9 Hz), 28.98 (s), 28.29 (s), 24.40 (s), 22.75 (s), 20.89 (s), 13.34 (s), 12.96 (s).

2β,3α-dihydroxy-5α-pregnan-20-one (1): To a solution of 2,3-epoxy-5α-pregnan-20-one (100 mg, 0.32 mmol) dissolved in THF (6 mL) was added 1N H₂SO₄ (0.16 mL, 0.8 mmol) and stirred for 24 h at room temperature. The reaction mixture was quenched with saturated NaHCO₃ solution and evaporated to fifth initial volume, diluted with water, and extracted with ethyl acetate (3×10 mL). Combined organic extracts were washed with water, dried over Na₂SO₄. The crude extract were subjected to flash column chromatography (n-hexane:EtOAc, 3:1). Concentration of the appropriate fractions in vacuo afforded 2β,3α-dihydroxy-5α-pregnan-20-one (1, 80 mg, 75%) 1H NMR (400 MHZ, CDCl₃) δ 3.97-3.86 (m, 2H), 2.54 (t, J=8.9 Hz, 1H), 2.13 (s, 4H), 2.03 (dt, J=11.4, 3.1 Hz, 1H), 1.92 (ddd, J=14.4, 13.1, 3.2 Hz, 1H), 1.81-1.55 (m, 5H), 1.49-1.12 (m, 8H), 1.07-0.88 (m, 4H), 0.85-0.73 (m, 1H), 0.63 (s, 3H). 13C NMR (101 MHZ, CDCl₃) δ 209.70 (s), 71.75 (s), 70.59 (s), 63.82 (s), 56.68 (s), 55.00 (s), 44.29 (s), 40.51 (s), 39.02 (d, J=16.5 Hz), 35.80 (s), 34.92 (s), 31.96-31.43 (m), 28.08 (s), 24.36 (s), 22.77 (s), 20.89 (s), 14.54 (s), 13.52 (s).

2α,3α-dihydroxy-5α-pregnan-20-one (2): To a solution of 2,3-ene-5α-pregnan-20-one and its Δ3 isomer (100 mg, 0.33 mmol) in tert-butanol-tetrahydrofuran-water (10:3:1, 3 mL) was added N-methylmorpholine-N-oxide (44 mg, 0.38 mmol) and osmium tetroxide (4 mg) and stirred at room temperature overnight. Then, the reaction was quenched by addition of an aqueous saturated solution of sodium bisulfite (3 mL). The mixture was extracted with ethyl acetate and washed with aqueous saturated solution of sodium bisulfite, brine and dried with Na₂SO₄. Solvent was evaporated and crude extract was subjected to flash column chromatography (n-hexane:EtOAc , 3:1). Concentration of the appropriate fractions in vacuo afforded 2β,3α-dihydroxy-5α-pregnan-20-one (1, 77.2 mg 70%) as white solid. 1H NMR (400 MHZ, CDCl₃) δ 3.99 (q, J=2.7 Hz, 1H), 3.84-3.74 (m, 1H), 2.55 (t, J=8.9 Hz, 1H), 2.13 (s, 4H), 2.10-1.91 (m, 4H), 1.78-1.09 (m, 16H), 1.05-0.78 (m, 5H), 0.62 (s, 3H). 13C NMR (101 MHZ, CDCl₃) δ 209.78 (s), 69.13 (d, J=19.1 Hz), 63.78 (s), 56.60 (s), 54.06 (s), 44.24 (s), 40.92 (s), 38.99 (s), 38.14 (s), 36.95 (s), 35.12 (s), 34.80 (s), 34.20 (s), 31.78 (s), 31.55 (s), 29.71 (s), 27.54 (s), 24.39 (s), 22.79 (s), 20.93 (s), 13.48 (s), 12.42 (s).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Adams S M, et al. ER-β mediates 17β-estradiol attenuation of HIV-1 Tat-induced apoptotic signaling. Synapse. 2010 November; 64 (11): 829-38.
2. Adams S M, et al. Soy isoflavones genistein and daidzein exert anti-apoptotic actions via a selective ER-mediated mechanism in neurons following HIV-1 Tat (1-86) exposure. PLOS One. 2012; 7 (5): e37540.
3. Agbottah E, et al. Inhibition of HIV-1 virus replication using small soluble Tat peptides. Virology. 2006 Feb. 20; 345 (2): 373-89.
4. Aoki, S.; et al. Cortistatins A, B, C, and D, Anti-Angiogenic Steroidal Alkaloids, from the Marine Sponge Corticium Simplex. J. Am. Chem. Soc. 2006.
5. Asin S N, et al. Estradiol and progesterone regulate HIV type 1 replication in peripheral blood cells. AIDS Res Hum Retroviruses. 2008 May; 24 (5): 701-16.
6. Avdoshina V, et al. The HIV Protein gp120 Alters Mitochondrial Dynamics in Neurons. Neurotox Res. 2016 May; 29 (4): 583-593.
7. Balinang J M, et al. Productive infection of human neural progenitor cells by R₅ tropic HIV-1: opiate co-exposure heightens infectivity and functional vulnerability. AIDS. 2017 Mar. 27; 31 (6): 753-764.
8. Barak O, et al. Intracerebral HIV-1 glycoprotein 120 produces sickness behavior and pituitary-adrenal activation in rats: role of prostaglandins. Brain Behav Immun. 2002 December; 16 (6): 720-35.
9. Barbaccia M L, et al. The effects of inhibitors of GABAergic transmission and stress on brain and plasma allopregnanolone concentrations. Br J Pharmacol. 1997 April; 120 (8): 1582-8.
10. Bauer M E, et al. Inflammation in psychiatric disorders: what comes first? Ann N Y Acad Sci. 2019 February; 1437 (1): 57-67.
11. Bertrand S J, et al. HIV-1 Tat and cocaine mediated synaptopathy in cortical and midbrain neurons is prevented by the isoflavone Equol. Front Microbiol. 2015 Sep. 8; 6:894.
12. Biggio G, et al. Allopregnanolone modulation of HPA axis function in the adult rat. Psychopharmacology (Berl). 2014 September; 231 (17): 3437-44.
13. Bing E G, et al. Psychiatric disorders and drug use among human immunodeficiency virus-infected adults in the United States. Arch Gen Psychiatry. 2001; 58:721-8.
14. Bons J et al. Adrenal disorders in human immunodeficiency virus (HIV) infected patients. Ann Endocrinol (Paris). 2013 December; 74 (5-6): 508-14.
15. Brant R, Sample Size Calculators, University of British Columbia, Accessed December 2019.
16. Brinton R D. Neurosteroids as regenerative agents in the brain: therapeutic implications. Nat Rev Endocrinol. 2013 April; 9 (4): 241-50.
17. Brooke S M, et al. Energy dependency of glucocorticoid exacerbation of gp120 neurotoxicity. J Neurochem. 1998; 71:1187-93.
18. Bruce-Keller A J, et al. Morphine causes rapid increases in glial activation and neuronal injury in the striatum of inducible HIV-1 Tat transgenic mice. Glia. 2008 October; 56 (13): 1414-27.
19. Camille Melon L, et al. GABAergic regulation of the HPA and HPG axes and the impact of stress on reproductive function. J Steroid Biochem Mol Biol. 2016 June; 160:196-203.
20. Carey A N, et al. Expression of HIV-Tat protein is associated with learning and memory deficits in the mouse. Behav Brain Res. 2012 Apr. 1; 229 (1): 48-56.
21. Chang H C, et al. HIV-1 Tat protein exits from cells via a leaderless secretory pathway and binds to extracellular matrix-associated heparan sulfate proteoglycans through its basic region. AIDS. 1997; 11:1421-31.
22. Chrousos G P, et al. Hypothalamic-pituitary-adrenal axis in HIV infection and disease. Endocrinol Metab Clin North Am. 2014 September; 43 (3): 791-806. doi: 10.1016/j.ecl.2014.06.002.
23. Cohen M S, et al; HPTN 052 Study Team. Prevention of HIV-1 infection with early antiretroviral therapy. N Engl J Med. 2011; 365:493-505.
24. Devadas K, et al. Modulation of HIV replication in monocyte derived macrophages (MDM) by steroid hormones. PLOS One. 2018 Jan. 26; 13 (1): e0191916.
25. Dirven B C J, et al. Epigenetic programming of the neuroendocrine stress response by adult life stress. J Mol Endocrinol. 2017 July; 59 (1): R₁₁—R₃₁.
26. Dreyer E B, et al. HIV-1 coat protein neurotoxicity prevented by calcium channel antagonists. Science. 1990; 248:364-7
27. Erdfelder E, et al. GPOWER: A general power analysis program. Behav Res Meth Instr Comput. 1996; 28:1-11.
28. Eugenin E A, et al. HIV-tat induces formation of an LRP-PSD-95-NMDAR-nNOS complex that promotes apoptosis in neurons and astrocytes. Proc Natl Acad Sci USA. 2007; 104:3438-43.
29. Fava G A, et al. Clinical characterization of allostatic overload. Psychoneuroendocrinology. 2019 October; 108:94-101.
30. Festa L K, et al. CXCL12-induced rescue of cortical dendritic spines and cognitive flexibility. Elife. 2020 Jan. 23; 9.
31. Fields J A, et al. HIV in the cART era and the mitochondrial: immune interface in the CNS. Int Rev Neurobiol. 2019; 145:29-65.
32. Fields J A, et al. Tenofovir disoproxil fumarate induces peripheral neuropathy and alters inflammation and mitochondrial biogenesis in the brains of mice. Sci Rep. 2019 Nov. 20; 9 (1): 17158.
33. Fischer S, et al. Cortisol as a predictor of psychological therapy response in anxiety disorders-Systematic review and meta-analysis. J Anxiety Disord. 2017 April; 47:60-68.
34. Fitting S, et al. Interactive HIV-1 Tat and morphine-induced synaptodendritic injury is triggered through focal disruptions in Na⁺ influx, mitochondrial instability, and Ca²⁺ overload. J Neurosci. 2014 Sep. 17; 34 (38): 12850-64.
35. Fitting S, et al. Opiate addiction therapies and HIV-1 Tat: interactive effects on glial [Ca²⁺]i, oxyradical and neuroinflammatory chemokine production and correlative neurotoxicity. Curr HIV Res. 2014; 12 (6): 424-34. PMCID: PMC4475822
36. Freda P U, et al. The hypothalamus-pituitary-adrenal axis in HIV disease. AIDS Read. 1999 January-February; 9 (1): 43-50.
37. Frye C A, et al. Prenatal Stress Alters Progestogens to Mediate Susceptibility to Sex-Typical, Stress-Sensitive Disorders, such as Drug Abuse: A Review. Front Psychiatry. 2011 Oct. 17; 2:52. PMCID: PMC3195272
38. Gallagher P, et al. Neuropsychological functioning in health and mood disorder: Modulation by glucocorticoids and their receptors. Psychoneuroendocrinology. 2009 December; 34 Suppl 1: S196-207.
39. George M M, et al. Human immune deficiency virus (HIV) infection and the hypothalamic pituitary adrenal axis. Rev Endocr Metab Disord. 2013 June; 14 (2): 105-12.
40. Godai K, et al. Ryanodine Receptor to Mitochondrial Reactive Oxygen Species Pathway Plays an Important Role in Chronic Human Immunodeficiency Virus gp120MN-Induced Neuropathic Pain in Rats. Anesth Analg. 2019 July; 129 (1): 276-286.
41. Gomes A C, et al. Hypogonadotropic hypogonadism in human immunodeficiency virus-infected men: uncommonly low testosterone levels. Endocrinol Diabetes Metab Case Rep. 2017 Sep. 6; 2017. pii: 17-0104.
42. Gonek M, et al. CCR5 mediates HIV-1 Tat-induced neuroinflammation and influences morphine tolerance, dependence, and reward. Brain Behav Immun. 2018 March; 69:124-138. PMCID: PMC5857418
43. Greenwood S M, et al. Dendritic and mitochondrial changes during glutamate excitotoxicity. Neuropharmacology. 2007 December; 53 (8): 891-8.
44. Guennoun R, et al. Progesterone and allopregnanolone in the central nervous system: response to injury and implication for neuroprotection. J Steroid Biochem Mol Biol. 2015 February; 146:48-61.
45. Gunn B G, et al. GABAA receptor-acting neurosteroids: a role in the development and regulation of the stress response. Front Neuroendocrinol. 2015 January; 36:28-48.
46. Hahn Y K, et al. Central HIV-1 Tat exposure elevates anxiety and fear conditioned responses of male mice concurrent with altered mu-opioid receptor-mediated G-protein activation and β-arrestin 2 activity in the forebrain. Neurobiol Dis. 2016; 92:124-36. PMCID: PMC4907901
47. Hahn Y K, et al. Effects of chronic HIV-1 Tat exposure in the CNS: heightened vulnerability of males versus females to changes in cell numbers, synaptic integrity, and behavior. Brain Struct Funct. 2015 March; 220 (2): 605-23.
48. Hammond E R, et al; CHARTER Group. Persistent CSF but not plasma HIV RNA is associated with increased risk of new-onset moderate-to-severe depressive symptoms; a prospective cohort study. J Neurovirol. 2016 August; 22 (4): 479-87.
49. Henderson L J, et al. Presence of Tat and transactivation response element in spinal fluid despite antiretroviral therapy. AIDS. 2019 Dec. 1; 33 Suppl 2: S145-S157.
50. Heron P M, et al. Estrogen receptor alpha inhibits the estrogen-mediated suppression of HIV transcription in astrocytes: implications for estrogen neuroprotection in HIV dementia. AIDS Res Hum Retroviruses. 2009 November; 25 (11): 1071-81.
51. Horak I D, et al. No T-cell tyrosine protein kinase signalling or calcium mobilization after CD4 association with HIV-1 or HIV-1 gp120. Nature. 1990 Dec. 6; 348 (6301): 557-60.
52. Hossain M I, et al. An efficient synthetic route to L-γ-methyleneglutamine and its amide derivatives, and their anticancer activity. Under consideration at ChemMedChem.
53. Hu XT. HIV-1 Tat-Mediated Calcium Dysregulation and Neuronal Dysfunction in Vulnerable Brain Regions. Curr Drug Targets. 2016; 17 (1): 4-14.
54. Hudson L, et al. Detection of the human immunodeficiency virus regulatory protein t at in CNS tissues. J Neurovirol. 2000; 6:145-55.
55. Irwin R W, et al. Allopregnanolone as regenerative therapeutic for Alzheimer's disease: translational development and clinical promise. Prog Neurobiol. 2014 February; 113:40-55.
56. Irwin R W, et al. Allopregnanolone Preclinical Acute Pharmacokinetic and Pharmacodynamic Studies to Predict Tolerability and Efficacy for Alzheimer's Disease. PLOS One. 2015; 10 (6): e0128313.
57. Irwin R W, et al. Frontiers in therapeutic development of allopregnanolone for Alzheimer's disease and other neurological disorders. Front Cell Neurosci. 2014 Jul. 30; 8:203.
58. Johansson I M, et al. Allopregnanolone inhibits learning in the Morris water maze. Brain Res. 2002; 934:125-131.
59. Jung-Testas I, et al. Steroid hormone receptors and steroid action in rat glial cells of the central and peripheral nervous system. J Steroid Biochem Mol Biol. 1998 April; 65 (1-6): 243-51.
60. Kalantari, P.; et al. 15-Deoxy-Δ 12'14-prostaglandin J 2 Inhibits HIV-1 Transactivating Protein, Tat, through Covalent Modification. FASEB J. 2009, 23 (8), 2366-2373.
61. Kendall S L, et al. Gonadal steroids differentially modulate neurotoxicity of HIV and cocaine: testosterone and ICI 182,780 sensitive mechanism. BMC Neurosci. 2005 Jun. 8; 6:40.
62. Kesby J P, et al. Methamphetamine Exposure Combined with HIV-1 Disease or gp120 Expression: Comparison of Learning and Executive Functions in Humans and Mice.

### Neuropsychopharmacology. 2015 July; 40 (8): 1899-909.

63. Kesby J P, et al; Translational Methamphetamine AIDS Research Center (TMARC) Group. Cognitive deficits associated with combined HIV gp120 expression and chronic methamphetamine exposure in mice. Eur Neuropsychopharmacol. 2015 January; 25 (1): 141-50.
64. Kessing C F, et al. In Vivo Suppression of HIV Rebound by Didehydro-Cortistatin A, a “Block-and-Lock” Strategy for HIV-1 Treatment. Cell Rep. 2017 Oct. 17; 21 (3): 600-611.
65. Khan, M. I. H.; et al. Discovery of 5α-pregnan-2β, 3α-diol-20-one as a neuroHIV Protective Agent, University of Mississippi School of Pharmacy Annual Poster Session, Oct. 19, 2021 (accessed May 12, 2022).
66. Kim B O, et al. Neuropathologies in transgenic mice expressing human immunodeficiency virus type 1 Tat protein under the regulation of the astrocyte-specific glial fibrillary acidic protein promoter and doxycycline. Am J Pathol. 2003 May; 162 (5): 1693-707.
67. Kumar M, et al. HIV-1 infection and its impact on the HPA axis, cytokines, and cognition. Stress. 2003 September; 6 (3): 167-72.
68. Kumar M, et al. The HPA axis in HIV-1 infection. J Acquir Immune Defic Syndr. 2002 Oct. 1; 31 Suppl 2: S89-93.
69. Kutsch O, et al. Direct and quantitative single-cell analysis of human immunodeficiency virus type 1 reactivation from latency. J Virol. 2002 September; 76 (17): 8776-86.
70. Lachâtre M, et al. HIV and hypogonadism: a new challenge for young-aged and middle-aged men on effective antiretroviral therapy. AIDS. 2017 Jan. 28; 31 (3): 451-453. doi: 10.1097/QAD.0000000000001348.
71. Langford D, et al. Doxycycline-inducible and astrocyte-specific HIV-1 Tat transgenic mice (iTat) as an HIV/neuroAIDS model. J Neurovirol. 2018 April; 24 (2): 168-179.
72. Leibrand C R, et al. HIV-1 Tat and opioids act independently to limit antiretroviral brain concentrations and reduce blood-brain barrier integrity. J Neurovirol. 2019; 25:560-577. PMCID: PMC6750988
73. Li C, et al. Tat inhibition by didehydro-Cortistatin A promotes heterochromatin formation at the HIV-1 long terminal repeat. Epigenetics Chromatin. 2019 Apr. 16; 12 (1): 23.
74. Li W, et al. NMDA receptor activation by HIV-Tat protein is clade dependent. J Neurosci. 2008 Nov. 19; 28 (47): 12190-8.
75. Lipton S A. HIV-related neurotoxicity. Brain Pathol. 1991 April; 1 (3): 193-9.
76. Luchetti S, et al. Neurosteroid and GABA-A receptor alterations in Alzheimer's disease, Parkinson's disease and multiple sclerosis. Neuroscience. 2011 Sep. 15; 191:6-21.
77. Marks W D, et al. HIV-1 Tat causes cognitive deficits and selective loss of parvalbumin, somatostatin, and neuronal nitric oxide synthase expressing hippocampal CA1 interneuron subpopulations. J Neurovirol. 2016; 22:747-762. PMCID: PMC5107352
78. Marques A H, et al. Glucocorticoid dysregulations and their clinical correlates. From receptors to therapeutics. Ann N Y Acad Sci. 2009 October; 1179:1-18.
79. Masvekar R R, et al. GSK3β-activation is a point of convergence for HIV-1 and opiate-mediated interactive neurotoxicity. Mol Cell Neurosci. 2015 March; 65:11-20.
80. Masvekar R R, et al. Morphine enhances HIV-1SF162-mediated neuron death and delays recovery of injured neurites. PLOS One. 2014 Jun. 20; 9 (6): e100196.
81. Mattson M P, et al. Cell death in HIV dementia. Cell Death Differ. 2005; 12:893-904.
82. Mclaughlin J P, et al. Conditional Human Immunodeficiency Virus Transactivator of Transcription Protein Expression Induces Depression-like Effects and Oxidative Stress. Biol Psychiatry Cogn Neurosci Neuroimaging. 2017; 2:599-609. PMCID: PMC5648358
83. McLaurin K A, et al. Selective Estrogen Receptor β Agonists: a Therapeutic Approach for HIV-1 Associated Neurocognitive Disorders. J Neuroimmune Pharmacol. 2019; In press. doi: 10.1007/s11481-019-09900-y.
84. Mediouni S, et al. Didehydro-Cortistatin A Inhibits HIV-1 by Specifically Binding to the Unstructured Basic Region of Tat. mBio. 2019 Feb. 5; 10 (1). pii: e02662-18.
85. Mediouni S, et al. Didehydro-cortistatin A inhibits HIV-1 Tat mediated neuroinflammation and prevents potentiation of cocaine reward in Tat transgenic mice. Curr HIV Res. 2015; 13:64-79. PMCID: PMC4416414
86. Mediouni S, et al. The Tat inhibitor didehydro-cortistatin A suppresses SIV replication and reactivation. FASEB J. 2019 July; 33 (7): 8280-8293.
87. Melcangi R C, et al. Allopregnanolone: state of the art. Prog Neurobiol. 2014 February; 113:1-5.
88. Meltzer-Brody S, et al. Brexanolone injection in post-partum depression: two multicentre, double-blind, randomised, placebo-controlled, phase 3 trials. Lancet. 2018 Sep. 22; 392 (10152): 1058-1070.
89. Micevych P, et al. Synthesis and function of hypothalamic neuroprogesterone in reproduction. Endocrinology. 2008 June; 149 (6): 2739-42.
90. Mirza F S, et al. Endocrinological aspects of HIV infection. J Endocrinol Invest. 2018 August; 41 (8): 881-899.
91. Mocchetti I, et al. Neurotoxicity of human immunodeficiency virus-1: viral proteins and axonal transport. Neurotox Res. 2012 January; 21 (1): 79-89.
92. Montefiori D C. Measuring HIV neutralization in a luciferase reporter gene assay. Methods Mol Biol. 2009; 485:395-405
93. Mousseau G, et al. An analog of the natural steroidal alkaloid cortistatin A potently suppresses Tat-dependent HIV transcription. Cell Host Microbe. 2012 Jul. 19; 12 (1): 97-108.
94. Mousseau G, et al. Resistance to the Tat Inhibitor Didehydro-Cortistatin A Is Mediated by Heightened Basal HIV-1 Transcription. mBio. 2019 Jul. 2; 10 (4). pii: e01750-18.
95. Mousseau G, et al. The Tat Inhibitor Didehydro-Cortistatin A Prevents HIV-1 Reactivation from Latency. mBio. 2015 Jul. 7; 6 (4): e00465.
96. Mukerji S S, et al. Low Neuroactive Steroids Identifies a Biological Subtype of Depression in Adults with Human Immunodeficiency Virus on Suppressive Antiretroviral Therapy. J Infect Dis. 2020 Mar. 10. pii: jiaa104.
97. Muñoz L D, et al. Progesterone inhibits HIV-1 replication in human trophoblast cells through inhibition of autocrine tumor necrosis factor secretion. J Infect Dis. 2007 May 1; 195 (9): 1294-302.
98. Napier T C, et al. Repeated cocaine treatment enhances HIV-1 Tat-induced cortical excitability via over-activation of L-type calcium channels. J Neuroimmune Pharmacol. 2014 June; 9 (3): 354-68.
99. Narayan, V.; et al. Celastrol Inhibits Tat-Mediated Human Immunodeficiency Virus (HIV) Transcription and Replication. J. Mol. Biol. 2011, 410 (5), 972-983.
100. Nath A, et al. Neurotoxicity and dysfunction of dopaminergic systems associated with AIDS dementia. J Psychopharmacol. 2000; 14:222-7.
101. Nickisch K, et al. Synthesis and biological evaluation of partially fluorinated antiprogestins and mesoprogestins. Steroids. 2013 February; 78 (2): 255-67.
102. Norman J P, et al. HIV-1 trans activator of transcription protein elicits mitochondrial hyperpolarization and respiratory deficit, with dysregulation of complex IV and nicotinamide adenine dinucleotide homeostasis in cortical neurons. J Immunol. 2007; 178:869-76.
103. Parada C A, et al. A novel RNA polymerase II-containing complex potentiates Tat-enhanced HIV-1 transcription. EMBO J. 1999; 18:3688-701.
104. Paris J J, et al. 5α-reduced progestogens ameliorate mood-related behavioral pathology, neurotoxicity, and microgliosis associated with exposure to HIV-1 Tat. Brain Behav Immun. 2016; 55:202-214. PMCID: PMC4899138
105. Paris J J, et al. Anxiety-like behavior of mice produced by conditional central expression of the HIV-1 regulatory protein, Tat. Psychopharmacology (Berl). 2014b; 231:2349-60. PMCID: PMC4020990
106. Paris J J, et al. Exposure to HIV-1 Tat in brain impairs sensorimotor gating and activates microglia in limbic and extralimbic brain regions of male mice. Behav Brain Res. 2015; 291:209-18. PMCID: PMC4497922
107. Paris J J, et al. Immune stress in late pregnant rats decreases length of gestation and fecundity, and alters later cognitive and affective behaviour of surviving pre-adolescent offspring. Stress. 2011 November; 14 (6): 652-64. PMCID: PMC3376536
108. Paris J J, et al. Pregnane steroidogenesis is altered by HIV-1 Tat and morphine: Physiological allopregnanolone is protective against neurotoxic and psychomotor effects. Neurobiol Stress. 2020 Jan. 29; 12:100211. PMCID: PMC7109513
109. Paris J J, et al. Progesterone protects normative anxiety-like responding among ovariectomized female mice that conditionally express the HIV-1 regulatory protein, Tat, in the CNS. Horm Behav. 2014a; 65:445-53. PMCID: PMC4067900
110. Paris J J, et al. Sex differences in salivary cortisol in response to acute stressors among healthy participants, in recreational or pathological gamblers, and in those with posttraumatic stress disorder. Horm Behav. 2010; 57:35-45. PMCID: PMC2858325
111. Patchev V K, et al. The neurosteroid tetrahydroprogesterone attenuates the endocrine response to stress and exerts glucocorticoid-like effects on vasopressin gene transcription in the rat hypothalamus. Neuropsychopharmacology. 1996 December; 15 (6): 533-40.
112. Patchev V K, et al. The neurosteroid tetrahydroprogesterone counteracts corticotropin-releasing hormone-induced anxiety and alters the release and gene expression of corticotropin-releasing hormone in the rat hypothalamus. Neuroscience. 1994 September; 62 (1): 265-71.
113. Perez S, et al. Persistence of SIV in the brain of SIV-infected Chinese rhesus macaques with or without antiretroviral therapy. J Neurovirol. 2018 February; 24 (1): 62-74.
114. Perry S W, et al. Adjunctive therapies for HIV-1 associated neurologic disease. Neurotox Res. 2005; 8:161-6.
115. Purohit V, et al. Drugs of abuse, dopamine, and HIV-associated neurocognitive disorders/HIV-associated dementia. Mol Neurobiol. 2011 August; 44 (1): 102-10.
116. Raber J, et al. Central nervous system expression of HIV-1 Gp120 activates the hypothalamic-pituitary-adrenal axis: evidence for involvement of NMDA receptors and nitric oxide synthase. Virology. 1996 Dec. 15; 226 (2): 362-73.
117. Reddy D S, et al. Clinical Potential of Neurosteroids for CNS Disorders. Trends Pharmacol Sci. 2016 July; 37 (7): 543-561.
118. Reddy S D. Sex differences in the anticonvulsant activity of neurosteroids. J Neurosci Res. 2017 Jan. 2; 95 (1-2): 661-670.
119. Robel P, et al. Biosynthesis and assay of neurosteroids in rats and mice: functional correlates. J Steroid Biochem Mol Biol. 1995 June; 53 (1-6): 355-60.
120. Robel P, et al. Neurosteroids: biosynthesis and function. Crit Rev Neurobiol. 1995; 9 (4): 383-94.
121. Rochira V, et al. Hypogonadism in the HIV-infected man. Endocrinol Metab Clin North Am. 2014 September; 43 (3): 709-30. doi: 10.1016/j.ecl.2014.06.005.
122. Rodriguez-Garcia M, et al. Estradiol reduces susceptibility of CD4+ T cells and macrophages to HIV-infection. PLOS One. 2013 Apr. 17; 8 (4): e62069.
123. Rosenblat J D, et al. Inflammation as a neurobiological substrate of cognitive impairment in bipolar disorder: Evidence, pathophysiology and treatment implications. J Affect Disord. 2015 Dec. 1; 188:149-59.
124. Saddique F A, et al. Recent trends in ring opening of epoxides by amines as nucleophiles. Synth Comm Rev. 2016; 46 (10): 831-68.
125. Salahuddin M F, et al. Combined HIV-1 Tat and oxycodone activate the hypothalamic-pituitary-adrenal and-gonadal axes and promote psychomotor, affective, and cognitive dysfunction in female mice. Horm Behav. 2020 March; 119:104649. PMCID: PMC7071558
126. Salahuddin, M. F. et al. Allopregnanolone and NeuroHIV: Potential Benefits of Neuroendocrine Modulation in the Era of Antiretroviral Therapy. J. Neuroendocrinol. 2022, 34 (2), e13047.
127. Samikkannu T, et al. HIV-1 gp120 and morphine induced oxidative stress: role in cell cycle regulation. Front Microbiol. 2015 Jun. 23; 6:614.
128. Sayeed I, et al. Progesterone as a neuroprotective factor in traumatic and ischemic brain injury. Prog Brain Res. 2009; 175:219-37.
129. Saylor D, et al. HIV-associated neurocognitive disorder—pathogenesis and prospects for treatment. Nat Rev Neurol. 2016 April; 12 (4): 234-48.
130. Schier C J, et al. Selective Vulnerability of Striatal D2 versus D1 Dopamine Receptor-Expressing Medium Spiny Neurons in HIV-1 Tat Transgenic Male Mice. J Neurosci. 2017; 37:5758-5769. PMCID: PMC5469310
131. Silverstein P S, et al. HIV-1 gp120 and drugs of abuse: interactions in the central nervous system. Curr HIV Res. 2012 July; 10 (5): 369-83.
132. Soria V, et al. Targeting hypothalamic-pituitary-adrenal axis hormones and sex steroids for improving cognition in major mood disorders and schizophrenia: a systematic review and narrative synthesis. Psychoneuroendocrinology. 2018 July; 93:8-19.
133. Stern Y, et al. Multidisciplinary baseline assessment of homosexual men with and without human immunodeficiency virus infection. III. Neurologic and neuropsychological findings. Arch Gen Psychiatry. 1991 February; 48 (2): 131-8.
134. Sundar S K, et al. Human immunodeficiency virus glycoprotein (gp120) infused into rat brain induces interleukin 1 to elevate pituitary-adrenal activity and decrease peripheral cellular immune responses. Proc Natl Acad Sci USA. 1991 Dec. 15; 88 (24): 11246-50.
135. Sze Y, et al. Sex, stress and steroids. Eur J Neurosci. 2019 Nov. 8. doi: 10.1111/ejn. 14615.
136. Szotek E L, et al. 17β-Estradiol inhibits HIV-1 by inducing a complex formation between β-catenin and estrogen receptor α on the HIV promoter to suppress HIV transcription. Virology. 2013 Sep. 1; 443 (2): 375-83.
137. Timby E, et al. Pharmacokinetic and behavioral effects of allopregnanolone in healthy women. Psychopharmacology (Berl). 2006 June; 186 (3): 414-24.
138. Toggas S M, et al. Central nervous system damage produced by expression of the HIV-1 coat protein gp120 in transgenic mice. Nature. 1994 Jan. 13; 367 (6459): 188-93.
139. Turchan J, et al. Estrogen protects against the synergistic toxicity by HIV proteins, methamphetamine and cocaine. BMC Neurosci. 2001; 2:3.
140. Türkmen S, et al. Persistence of tolerance to the anaesthetic effect of allopregnanolone in male rats. Eur J Pharmacol. 2008 Sep. 11; 592 (1-3): 73-80.
141. Uchida M, et al. Inhibition by 9alpha-fluoromedoroxyprogesterone acetate (FMPA) against mammary carcinoma induced by dimethylbenz [a]anthracene in rats and angiogenesis in the rabbit cornea-comparison with medroxyprogesterone acetate (MPA). Cancer Lett. 2000 Jun. 1; 154 (1): 63-9.
142. Wallet C, et al. Microglial Cells: The Main HIV-1 Reservoir in the Brain. Front Cell Infect Microbiol. 2019 Oct. 24; 9:362.
143. Wan, Z.; et al. Triptolide Inhibits Human Immunodeficiency Virus Type 1 Replication by Promoting Proteasomal Degradation of Tat Protein. Retrovirology 2014, 11 (1), 88.
144. Wayman W N, et al. Enduring cortical alterations after a single in-vivo treatment of HIV-1 Tat. Neuroreport. 2012 Oct. 3; 23 (14): 825-9.
145. Wesselingh S L, et al. Intracerebral cytokine messenger RNA expression in acquired immunodeficiency syndrome dementia. Ann Neurol. 1993; 33:576-82.
146. Wiley C A, et al. Expression of HIV regulatory and structural mRNA in the central nervous system. AIDS. 1996; 10:843-7.
147. Wilson M E, et al. Estradiol negatively regulates HIV-LTR promoter activity in glial cells. AIDS Res Hum Retroviruses. 2006 April; 22 (4): 350-6.
148. Woods S P, et al. Cognitive neuropsychology of HIV-associated neurocognitive disorders. Neuropsychol Rev. 2009 June; 19 (2): 152-68.
149. Zapanti E, et al. Dysfunction of the hypothalamic-pituitary-adrenal axis in HIV infection and disease. Hormones (Athens). 2008 July-September; 7 (3): 205-16
150. Zhu D, et al. Dynamic aspects of acute tolerance to allopregnanolone evaluated using anaesthesia threshold in male rats. Br J Anaesth. 2004 October; 93 (4): 560-7.
151. Zou S, et al. Effects of HIV-1 Tat on oligodendrocyte viability are mediated by CaMKIIβ-GSK3β interactions. J Neurochem. 2019; 149:98-110. PMCID: PMC6438763

1. A compound comprising a structure of Formula I 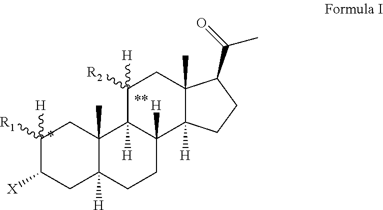
wherein R₁ and R₂ individually are H, halogen, CF₃, CH₂CF₃, OH, SH, OR₃, SR₃, substituted or unsubstituted C3-C7 aryl or heteroaryl, amino, C1-C10 alkylamino, C1-C10 dialkylamino, acyl, C1-C10 alkyl ester, or any combination thereof;
wherein R₃ is linear or branched C1-C10 alkyl, CF₃, CH₂CF₃, substituted or unsubstituted C3-C7 aryl or heteroaryl, or any combination thereof; and
wherein X is OR₄;
wherein R₄ is H; or
wherein R₁ and X together are an oxygen atom, forming an epoxide;
provided R₁ and R₂ are not both H.

2. The compound of claim 1, wherein R₁ and R₂ individually are selected from H, OH, dimethylamino, OCF₃, OCH₃, OCH₂CF₃, NH₂, or any combination thereof;
provided R₁ and R₂ are not both H.

3. The compound of claim 1, wherein when R₁ is not H, the structure of Formula I has substantially R stereochemistry, substantially S stereochemistry, or a mixture thereof at the carbon atom indicated by *.

4. The compound of claim 1, wherein when R₁ is not H, the carbon atom indicated by * has from about 5% to about 95% R stereochemistry and from about 95% to about 5% S stereochemistry.

5. The compound of claim 1, wherein when R₂ is not H, the structure of Formula I has substantially R stereochemistry, substantially S stereochemistry, or a mixture thereof at the carbon atom indicated by **.

6. The compound of claim 1, wherein when R₂ is not H, the carbon atom indicated by ** has from about 5% to about 95% R stereochemistry and from about 95% to about 5% S stereochemistry.

7. The compound of claim 1, wherein the structure of Formula I is selected from 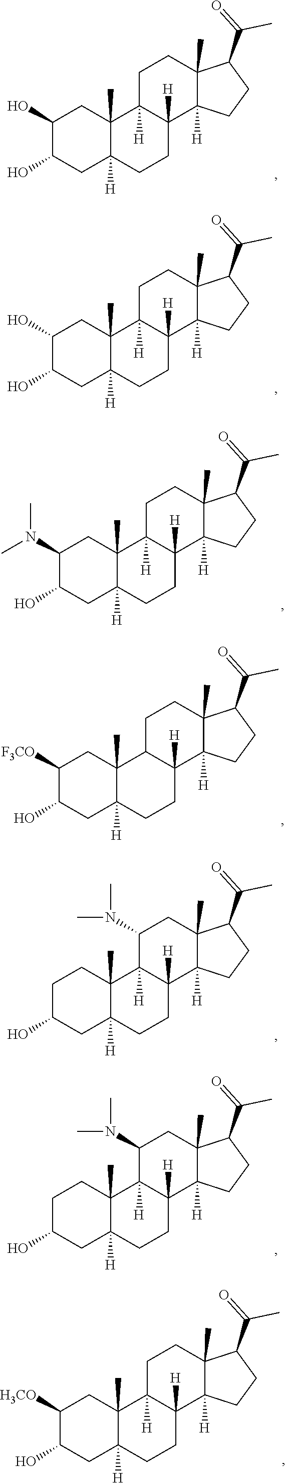 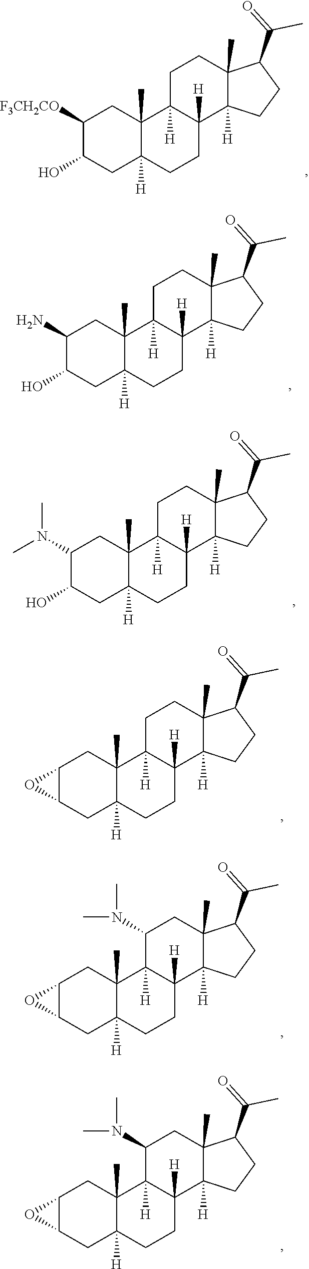
or any combination thereof.

8. A pharmaceutical composition comprising a therapeutically effective amount of the compound of claim 1 or a pharmaceutically acceptable salt thereof.

9. A method for treating HIV or neuroHIV in a subject, the method comprising administering the compound of claim 1 to the subject.

10. The method of claim 9, wherein administering the compound comprises administering from about 1 mg to about 100 mg of the compound per kg of body weight of the subject.

11. The method of claim 9, wherein the compound is administered orally, intravenously, subcutaneously, or via an implanted device.

12. The method of claim 9, wherein the compound is administered daily.

13. The method of claim 9 wherein the subject is a human.

14. The method of claim 9, wherein administering the compound to the subject reduces at least one symptom associated with HIV or neuroHIV in the subject.

15. The method of claim 14, wherein the at least one symptom comprises a neurocognitive disorder, a neuropsychiatric disorder, a deficit in learning or memory, behavioral inhibition, affective well-being, hypothalamic-pituitary-adrenal (HPA) stress-axis dysfunction, hypothalamic-pituitary-gonadal (HPG) axis dysfunction, hypothalamic-pituitary-thyroid (HPT) axis dysfunction, central nervous system viremia, elevated glucocorticoid levels, adrenal insufficiency, gonadal hormone insufficiency, glucocorticoid insensitivity, anxiety, depression, neurotoxicity of HIV medications, or any combination thereof.

16. The method of claim 9, wherein administering the compound to the subject inhibits production or activity of at least one HIV-associated protein.

17. The method of claim 16, wherein the at least one HIV-associated protein comprises trans-activator of transcription protein (Tat), envelope protein gp41, envelope protein gp120, p55, p24, p17, p6, p7, Rev, negative factor (Nef), viral protein R (Vpr), viral protein U (Vpu), virion infectivity factor (Vif), or any combination thereof.

18. The method of claim 9, further comprising administering at least one additional HIV treatment to the subject.

19. The method of claim 18, wherein the at least one additional HIV treatment comprises combination antiretroviral therapy (cART).

20. The method of claim 19, wherein the compound of Formula I reduces cytotoxicity of CART relative to administration of cART without performing the method.
